(19) United States
(12) Patent Application Publication
(10) Pub. No.: US 2025/0375461 A1
(43) Pub. Date: Dec. 11, 2025

(54) IDENTIFICATION OF A NEW POTENT INHIBITOR OF MYCOBACTERIAL GROWTH

(71) Applicants: Brown University, Providence, RI (US); Bryant University, Smithfield, RI (US)

(72) Inventors: Amit Basu, Barrington, RI (US); Christopher Reid, North Kingstown, RI (US); Jameson Pommenville, North Attleboro, MA (US)

(21) Appl. No.: 19/222,957

(22) Filed: May 29, 2025

Related U.S. Application Data
(60) Provisional application No. 63/657,078, filed on Jun. 6, 2024.

(51) Int. Cl. A61K 31/7056; A61K 31/706; A61P 31/06; G01N 33/569

(52) U.S. Cl. CPC A61K 31/7056; A61K 31/706; A61P 31/06; G01N 33/5695; G01N 2800/26; G01N 2800/52

(57) ABSTRACT

Disclosed are methods for that can be effective for treating a Mycobacterium infection in a biological organism, in particular in humans, involve administering a medicinal formulation that includes specific bioactive compounds with N-acetylglucosamine or N-glycolylglucosamine and triazole. The compound interacts with the Mycobacterium to provide treatment. The method can be applied without detrimental toxicity to human subjects and is particularly relevant for tuberculosis infections. The formulation can be administered in various ways, including orally and intravenously, and may be provided in a dosage of, for example, about 0.5-100 mg/kg of body weight. Additionally, the method may include steps for diagnosing and monitoring the treatment of the infection.

### CROSS REFERENCE TO RELATED APPLICATIONS

This disclosure claims priority to U.S. Provisional Patent App. No. 63/657,078, filed 6 Jun. 2024, the entire contents of which are hereby incorporated by reference.

### STATEMENT OF GOVERNMENT INTEREST

This invention was made with government support under grant number 2009522, awarded by the National Science Foundation, and grant number P20 GM103430 awarded by the National Institutes of Health. The government has certain rights in the invention.

### FIELD OF THE INVENTION

The embodiments of the present invention relate new methods and treatments for treating and for inhibiting Mycobacterium bacterial infections.

### BACKGROUND OF THE INVENTION

Mycobacteria are a type of bacteria that can cause serious infections in humans, including tuberculosis and leprosy. These infections are challenging to treat due to the bacteria's complex cell wall, which makes them resistant to many common antibiotics. The rise of drug-resistant strains of mycobacteria has further complicated treatment efforts, necessitating the development of new therapeutic strategies. Traditional treatments often involve lengthy courses of multiple antibiotics, which can lead to significant side effects and contribute to the development of resistance.

The need for innovative treatments is critical as mycobacterial infections continue to pose a significant public health challenge worldwide. The development of new drugs that can effectively target mycobacteria without contributing to resistance is a key area of research. Small molecules that can penetrate the mycobacterial cell wall and disrupt its function offer a promising avenue for new treatments. These molecules must be carefully designed to ensure they are both effective against the bacteria and safe for human use. As researchers continue to explore new chemical compounds, the potential for breakthroughs in the treatment of mycobacterial infections remains high.

Tuberculosis (TB) is an infectious disease that most often affects the lungs and is caused by a Mycobacterium type (genus) of bacteria. It spreads through the air when infected people sneeze, cough, or spit. Treatment can require the use of multiple antibiotics over a long period of time. Antibiotic resistance is a serious growing problem, with increasing rates of multiple drug-resistant tuberculosis (MDR-TB). About a quarter of the global population is estimated to have been infected with TB bacteria. About 5-10% of people infected with TB will eventually get symptoms and develop TB disease. Those who are infected but not yet ill with the disease cannot transmit it. TB disease can be fatal without treatment. In some countries, the Bacille Calmette-Guérin (BCG) vaccine is given to babies or small children to prevent TB. The vaccine prevents TB outside of the lungs but not in the lungs.

MDR-TB is a form of TB typically caused by bacteria that do not respond to isoniazid and rifampicin treatments, which are first-line TB drugs. MDR-TB can be treatable and curable by using second-line drugs. However, second-line treatment options require extensive medicines that are expensive and toxic.

In some cases, more extensive drug resistance can develop. TB caused by bacteria that do not respond to the most effective second-line TB drugs can leave patients with very limited treatment options. MDR-TB remains a public health crisis and a health security threat. Only about 2 in 5 people with drug resistant TB accessed treatment in 2022.

A total of about 1.3 million people died from TB in 2022 (including 167,000 people with HIV). Worldwide, TB is the second leading infectious killer after COVID-19 (above HIV and AIDS). US$13 billion is needed annually for TB prevention, diagnosis, treatment and care to achieve the global target agreed at the 2018 UN high level-meeting on TB.¹

Ending the TB epidemic by 2030 is among the health targets of the United Nations Sustainable Development Goals (SDGs).¹ In the facts of the long-crisis, new methods of treating TB are urgently needed to save human lives.

### BRIEF SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In accordance with some embodiments, a method is provided for treating a Mycobacterium infection in a biological organism, in particular for human treatments to save human lives. The method involves administering a medicinal formulation that includes a bioactive compound comprising N-acetylglucosamine or N-glycolylglucosamine and triazole, or any small molecule disclosed herein, which interacts with the Mycobacterium to combat the infection. Additionally, a medicinal formulation is disclosed for treating a tuberculosis infection in a human or in a biological organism. This formulation also contains a bioactive compound with N-acetylglucosamine or N-glycolylglucosamine and triazole or any small molecule disclosed herein, designed to interact with and treat the tuberculosis caused by Mycobacterium.

Tuberculosis has been a plague to humans' lives since antiquity. Unfortunately, the disease continues to take more lives yearly than almost any other infection. Surprisingly, molecules discussed herein have shown advanced activities towards Mycobacteria. Thus, a major purpose of the disclosure is to save lives in the face of a long-felt but unmet need to address these infections, especially in the failure of other treatments as shown by MDR-TB.

In some embodiments, the technology disclosed herein can be briefly summarized by the following list of Features:

Feature 1: A method for treating an infection, disease, or disorder in a subject, the method comprising the step of administering a therapeutically effective amount of a small molecule of Formula 1, 2, 3, and/or 4 to a subject or to a subject in need thereof:

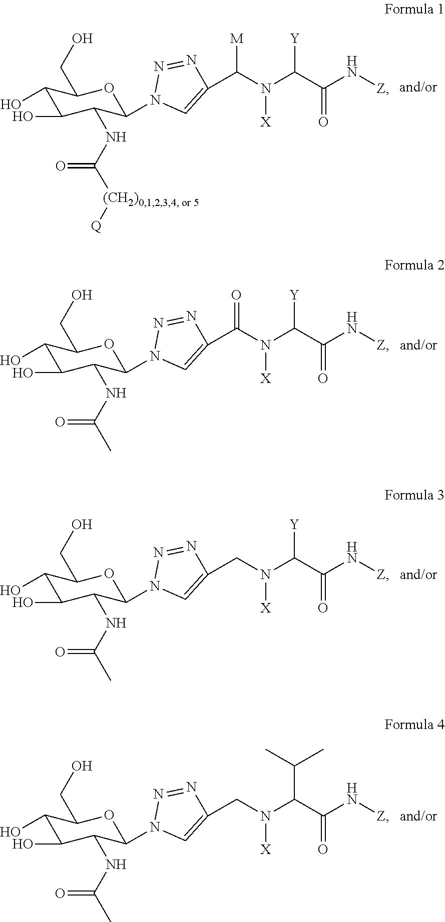

wherein any portion of a chemical structure represented by Formula 1, Formula 2, Formula 3, and/or Formula 4 can be inter-combined or interchanged; wherein each occurrence of Q, M, X, Y, and/or Z each independently includes or is each independently comprising a combination of —H, —OH, carbonyl (═O), F, N, —O—, —S—, —NH—, a halogen, methyl (—CH₃), t-butyl, —(CH₂)ₙ—CH₃, wherein independently each n=0, 1, 2, 3, 4, 5, 6, 7, 8, or 9; —O—(CH₂)ₙ—CH₃, wherein independently each n=0, 1, 2, 3, 4, 5, 6, 7, 8, or 9; —S—(CH₂)ₙ—CH₃, wherein independently each n=0, 1, 2, 3, 4, 5, 6, 7, 8, or 9; —NH—(CH₂)ₙ—CH₃, wherein independently each n=0, 1, 2, 3, 4, 5, 6, 7, 8, or 9; and/or a substituent shown below:

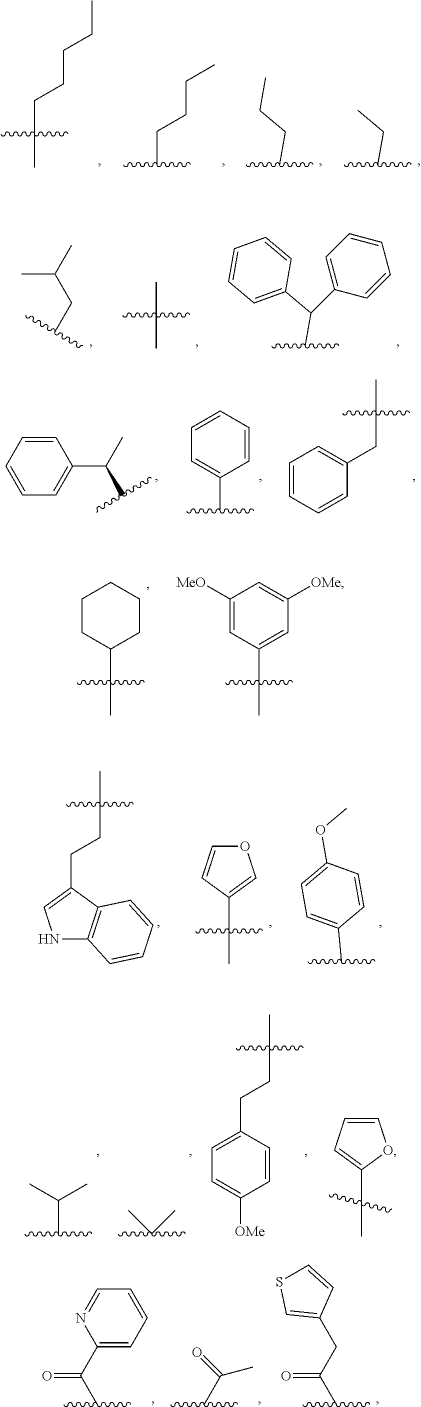

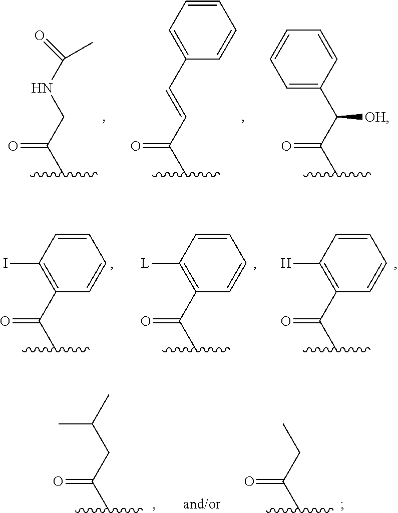

wherein each independent occurrence of [symbol] is a linker or a covalent bond to formula 1 and/or to any atom in formula 1 and represents an attachment to an atom as indicated in formula 1 and includes or is a combination of a single bond (−), a bond to a nitrogen, a bond to a carbon, a bond to an oxygen (—O—), a double bond (═), a triple bond (≡) or (—C₂H₂—), a bond to C—C, a bond to C—N, —(CH₂)ₙ—, wherein independently each n=0, 1, 2, 3, 4, 5, 6, 7, 8, or 9; or a combination thereof; and wherein each occurrence of Q, M, X, Y, and/or Z each independently can represent Q-Q, M-M, X-X, Y-Y, and/or Z-Z;
wherein each occurrence is independent of another occurrence and linked by a bond or by another linker [symbol] to any atom of the other; wherein each independent occurrence of L is a halogen including bromine, iodine, chlorine, or fluorine; and/or the step of administering a pharmaceutically acceptable salt thereof of any of the small molecules described above; and includes and/or the step of administering a pharmaceutically acceptable hydrate or solvate thereof, either with or without a salt form; and wherein any carbon in Formula 1, Formula 2, Formula 3, and/or Formula 4 can optionally be replaced by a boron or suitable atom configured to overcome a drug-resistance of a bacterial infection.

Feature 2 The method of feature 1, wherein the method includes a treating of a mycobacterium infection.

Feature 3: The method of feature 1, wherein the method includes a threating of mycobacterial infections to cover a range of infectious organisms including TB (tuberculosis) infections, non-TB infections, M. absescus complex, M. kansasii, M. xenopi, or a combination thereof.

Feature 4: The method of feature 1, the method comprising the step of administering a therapeutically effective amount of a small molecule including a chemical structure shown below to a subject; a pharmaceutically acceptable salt thereof, or administering a pharmaceutically acceptable hydrate or solvate thereof, either with or without a salt form:

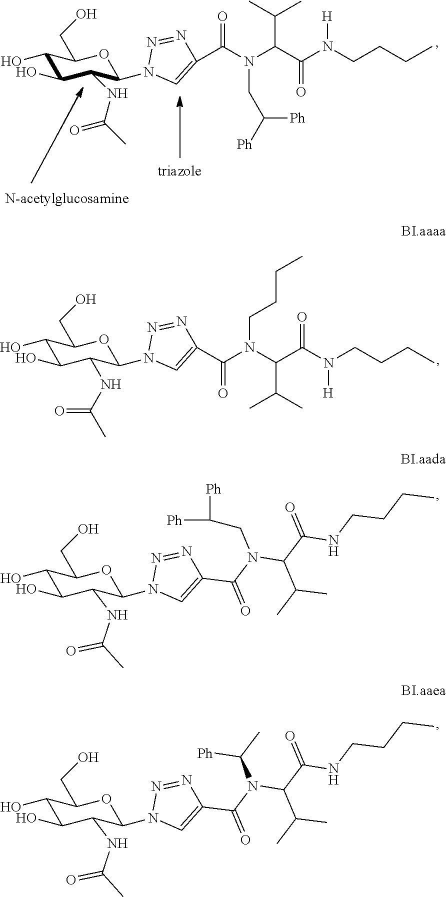

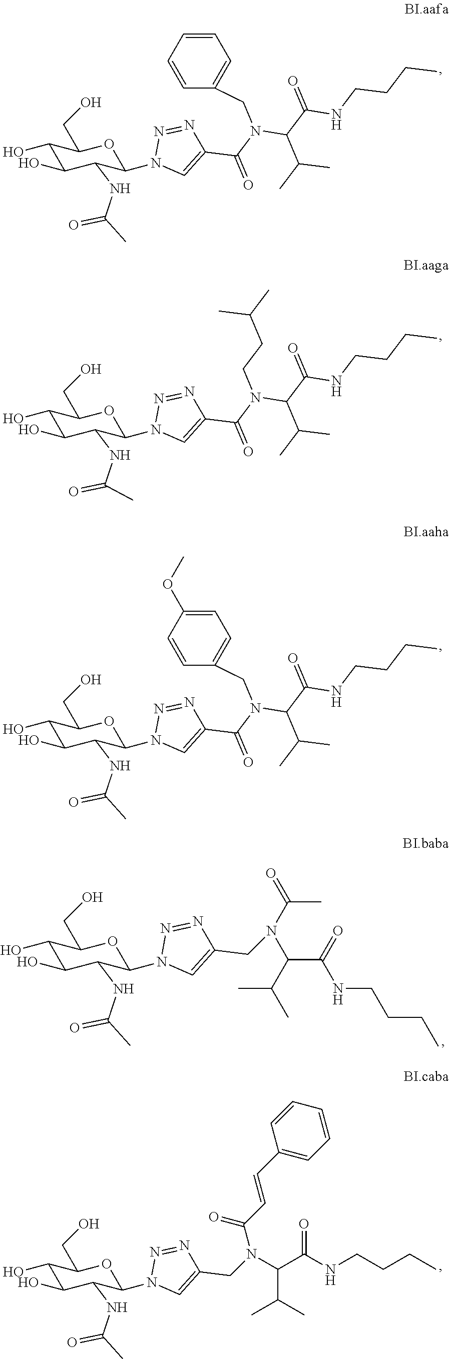 [SMILES: CCCCNC(=O)C(C(C)C)N(Cc1ccccc1)C(=O)c1cn(C2OC(CO)C(O)C(O)C2NC(C)=O)nn1 ; CCCCNC(=O)C(C(C)C)N(CCC(C)C)C(=O)c1cn(C2OC(CO)C(O)C(O)C2NC(C)=O)nn1 ; CCCCNC(=O)C(C(C)C)N(Cc1ccc(OC)cc1)C(=O)c1cn(C2OC(CO)C(O)C(O)C2NC(C)=O)nn1 ; CCCCNC(=O)C(C(C)C)N(Cc1cn(C2OC(CO)C(O)C(O)C2NC(C)=O)nn1)C(C)=O ; CCCCNC(=O)C(C(C)C)N(Cc1cn(C2OC(CO)C(O)C(O)C2NC(C)=O)nn1)C(=O)/C=C/c1ccccc1]

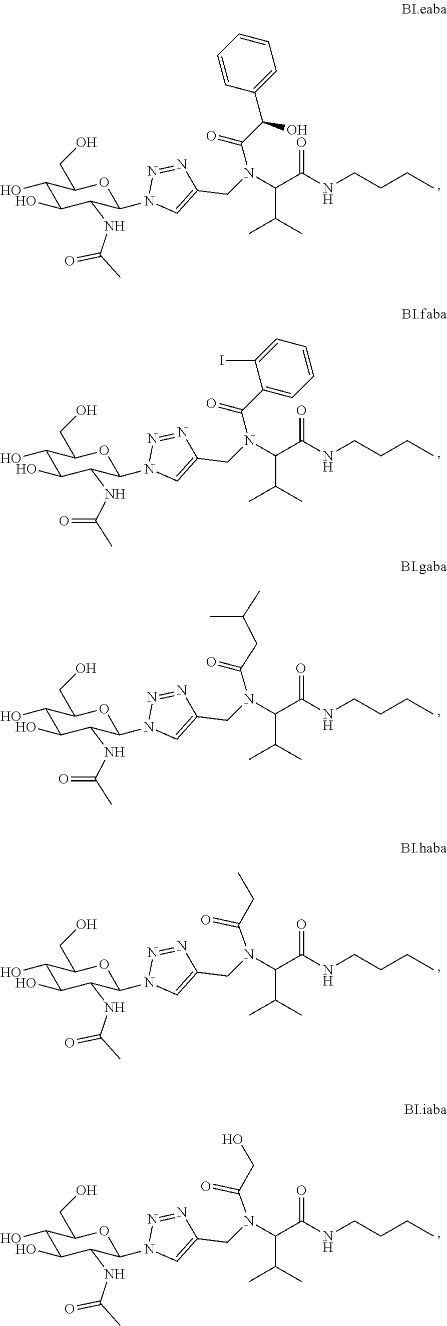 [SMILES: CCCCNC(=O)C(C(C)C)N(Cc1cn(C2OC(CO)C(O)C(O)C2NC(C)=O)nn1)C(=O)[C@H](O)c1ccccc1 ; CCCCNC(=O)C(C(C)C)N(Cc1cn(C2OC(CO)C(O)C(O)C2NC(C)=O)nn1)C(=O)c1ccccc1I ; CCCCNC(=O)C(C(C)C)N(Cc1cn(C2OC(CO)C(O)C(O)C2NC(C)=O)nn1)C(=O)CC(C)C ; CCCCNC(=O)C(C(C)C)N(Cc1cn(C2OC(CO)C(O)C(O)C2NC(C)=O)nn1)C(=O)CC ; CCCCNC(=O)C(C(C)C)N(Cc1cn(C2OC(CO)C(O)C(O)C2NC(C)=O)nn1)C(=O)CO]

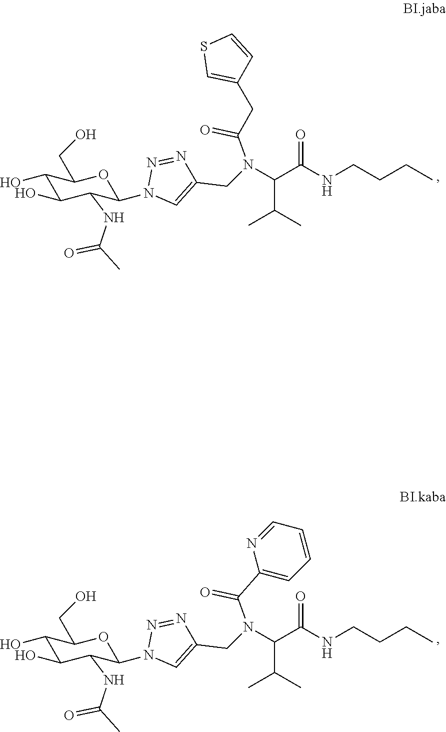 [SMILES: CCCCNC(=O)C(C(C)C)N(Cc1cn(C2OC(CO)C(O)C(O)C2NC(C)=O)nn1)C(=O)Cc1ccsc1 ; CCCCNC(=O)C(C(C)C)N(Cc1cn(C2OC(CO)C(O)C(O)C2NC(C)=O)nn1)C(=O)c1ccccn1]

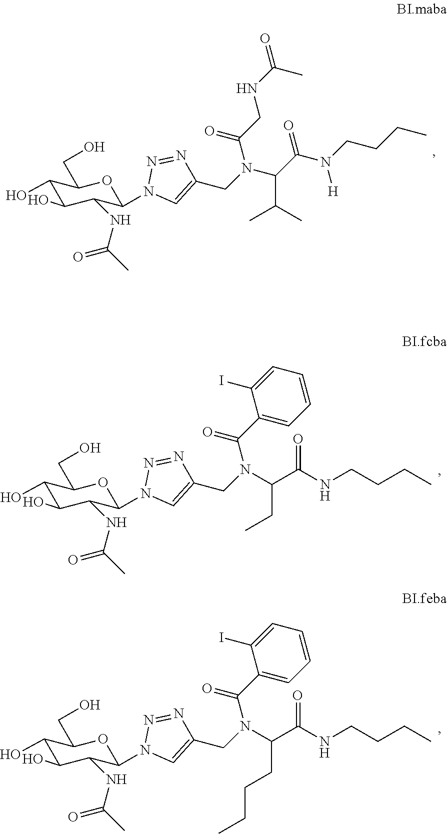 [SMILES: CCCCNC(=O)C(C(C)C)N(Cc1cn(C2OC(CO)C(O)C(O)C2NC(C)=O)nn1)C(=O)CNC(C)=O ; CCCCNC(=O)C(CC)N(Cc1cn(C2OC(CO)C(O)C(O)C2NC(C)=O)nn1)C(=O)c1ccccc1I ; CCCCNC(=O)C(CCCC)N(Cc1cn(C2OC(CO)C(O)C(O)C2NC(C)=O)nn1)C(=O)c1ccccc1I]

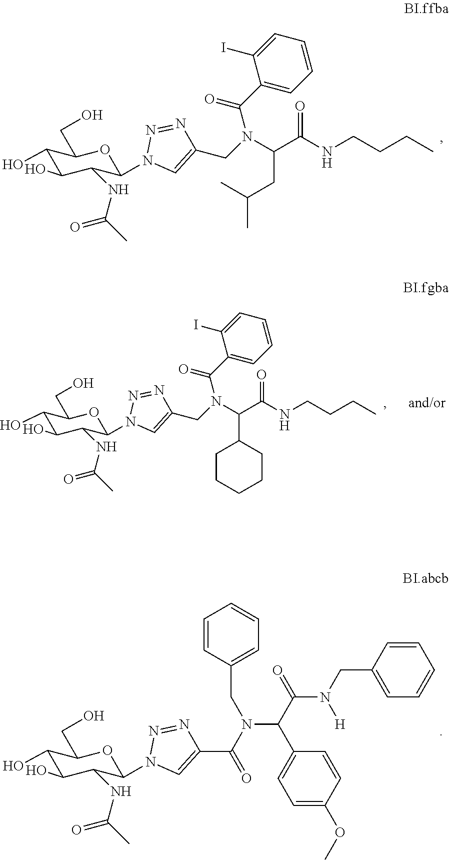 [SMILES: CCCCNC(=O)C(CC(C)C)N(Cc1cn(C2OC(CO)C(O)C(O)C2NC(C)=O)nn1)C(=O)c1ccccc1I ; CCCCNC(=O)C(C1CCCCC1)N(Cc1cn(C2OC(CO)C(O)C(O)C2NC(C)=O)nn1)C(=O)c1ccccc1I ; COc1ccc(C(C(=O)NCc2ccccc2)N(Cc2ccccc2)C(=O)c2cn(C3OC(CO)C(O)C(O)C3NC(C)=O)nn2)cc1]

Feature 5: The method of feature 3, wherein the infection includes a tuberculosis infection.

Feature 6: The method of any preceding feature, where a pharmaceutically acceptable salt includes alkyl, dialkyl, trialkyl or tetra-alkyl ammonium salts; L-arginine, benenthamine, benzathine, betaine, calcium hydroxide, choline, deanol, diethanolamine, diethylamine, 2-(diethylamino) ethanol, ethanolamine, ethylenediamine, N-methylglucamine, hydrabamine, 1H-imidazole, lithium, L lysine, magnesium, 4-(2-hydroxyethyl)-morpholine, piperazine, potassium, 1-(2-hydroxyethyl) pyrrolidine, sodium, triethanolamine, tromethamine, and zinc salts; Na, Ca, K, Mg, Zn or other metal salts; 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, I-ascorbic acid, I-aspartic acid, benzenesulfonic acid, benzoic acid, (+)-camphoric acid, (+) camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d glucoheptonic acid, d gluconic acid, d glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, I-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, I-pyroglutamic acid, salicylic acid, sebacic acid, stearic acid, succinic acid, sulfuric acid, I tartaric acid, thiocyanic acid, p-toluenesulfonic acid, trifluoroacetic acid, and/or undecylenic acid salt, and/or any pharmaceutically acceptable salt.

Feature 7: The method of any preceding feature, wherein a pharmaceutically acceptable salt can be present as a solvate optionally including water, methanol, ethanol, or dimethylformamide.

Feature 8: The method of any preceding feature, wherein the small molecule comprises a polymorph, a cocrystal, or an amorphous form.

Feature 9: The method of any preceding feature, wherein a solvate and/or a hydrate is formed by a slow evaporation whereby water and/or solvent remain hydrogen bonded with OH groups in the small molecule.

Feature 10: The method of feature 7, wherein a formation of a solvate/hydrate is confirmed after the evaporation by using attenuated total reflectance Fourier transform infra-red spectroscopy (ATR-IR) wherein the solid solvate/hydrate is directly placed on the instrument and the subsequent IR spectrum is compared to the IR spectrum of the solid non-solvate, non-hydrate.

Feature 11: The method of feature 10, wherein the comparison of the solvate and/or hydrate indicates an increased hydrogen bonding in an ATR-IR spectrum of the solvate and/or hydrate.

Feature 12: The method of feature 1 or feature 2, further comprising diagnosing a Mycobacterium infection in the subject.

Feature 13: The method of feature 1 or feature 2, further comprising monitoring a treatment of a Mycobacterium infection in the subject.

Feature 14: The method of any preceding feature, wherein the small molecule, salt, and/or solvate comprises any combination of the chemical structures shown below:

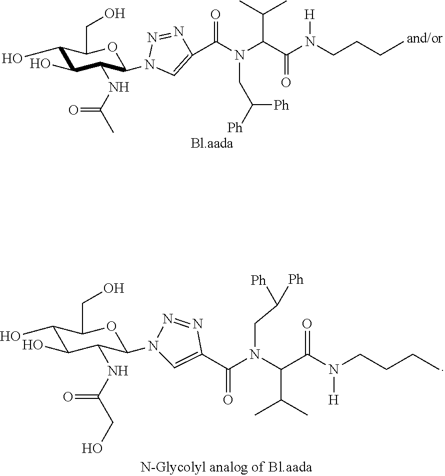

Feature 15: The method of feature 14, wherein BI.aada inhibits M. absescus with an MIC (minimum inhibitory concentration) of <500 nM.

Feature 16: The method of feature 14, wherein BI.aada inhibits M. smegmatis with an MIC of ≤50 nM, inhibits M. avium with an MIC of ≤500 nM, inhibits Gram negative E. coli WO0153 with an MIC of 70 UM, or a combination thereof.

Feature 17: The method of feature 14, wherein BI.aada has no activity inhibiting or against E. coli K2.

Feature 18: The method of any preceding feature, wherein the small molecule is in a form of a pharmaceutical composition.

Feature 19: The method of feature 18, wherein the pharmaceutical composition includes a polymer encapsulation.

Feature 20: The method of feature 19, wherein the polymer encapsulation improves an oral bioavailability and/or increase an uptake from a GI (gastrointestinal tract) presence.

Feature 21: The method of any preceding feature, further comprising wherein the method is utilized for a veterinary application or wherein the subject is not a human subject.

Feature 22: The pharmaceutical composition of feature 18, wherein the pharmaceutical composition is formulated for oral administration, parenteral administration, or administration via implanted reservoir.

Feature 23: The pharmaceutical composition of feature 18, wherein the pharmaceutical composition is stable at a room temperature of about 25 degrees Celsius and a humidity of about 50% for at least 3 months.

Feature 24: The method of feature 23, wherein the pharmaceutical composition is stable for over a year.

Feature 25: The pharmaceutical composition of feature 23, wherein the stability is an increase of not more than 0.5 percent of an impurity or a degradant; wherein an impurity or a degradant is any substance and/or a chemical that was not present at the beginning of the at least 3 months of the stability measurement.

Feature 26: The method of feature 1, further comprising wherein the method is a prophylactic method, a preventative method, or a method for a precaution against an infection.

Feature 27: The method of feature 26, wherein the subject is a normal healthy human subject.

Feature 28: The method of feature 18, wherein the pharmaceutical composition is administered with another therapeutic agent either at a same time or at a different time.

According to some aspects, the subject is a normal healthy human subject. In some embodiments, the methods herein further comprise diagnosing a Mycobacterium infection in the subject and/or further comprise monitoring the treatment of a Mycobacterium infection in the subject.

Other implementations are also described and recited herein. These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

Solely for the purpose of illustration, certain embodiments of the present invention are explained using examples in the drawings described below. It should be understood, however, that the invention is not limited to the precise arrangements, dimensions, and configurations shown. In the figures:

FIG. 1 shows microscopy of M. smegmatis in presence (right) and absence (left) of BI.aada

FIG. 2 shows microscopy of E. coli WO153 (leaky outer membrane) with control at left and 50 UM BI.aada at right.

FIG. 3 shows a MIC assay: E. coli WO154 (leaky strain) with BI.aada.

FIG. 4, FIG. 5, and FIG. 6 show enlarged views of MIC Assay: M. smegmatis with BI.aada.

FIG. 7 shows a repeat structure of peptidoglycan (A1-g chemotype) and cleavage sites of major bacterial autolysins. The box shows structures of bulgecin A and NAG-thiazoline, inhibitors of cell wall glycosyl hydrolases.

FIG. 8A shows a synthetic route for preparation of GNTs using copper accelerated azide-alkyne coupling.

FIG. 8B and FIG. 8C show structures of example GNTs synthesized and evaluated for antibacterial activity. FIG. 9A and FIG. 9B shows examples of GNTs with antibacterial activity against Bacillus.

FIG. 10A shows a dose dependent inhibition of pNP-GlcNAc hydrolysis by intact B. subtilis cells in the presence of BI.fgba confirming that the target is a GlcNAcase. FIG. 10B shows morphological changes to B. subtilis upon exposure to BI.fgba. Control cells (left) were grown in the presence of 1% DMSO and show the typical rod-shaped cells. Cells on the right were treated with 0.8×MIC (48 μM) BI.fgba and exhibit an elongated phenotype.

FIG. 11, shows a flow diagram of example steps of addressing a Mycobacterium infection.

FIG. 12 shows example steps of administering a medicinal formulation, according to some aspects.

It should be understood that while different numbers/numbering are/is sometimes used in some of the figures above to describe different embodiments and different aspects of the technology, any number from any figure can be inter-combined with a numbered aspect from any other figures. All trademarks, images, likenesses, words, and depictions in the drawings and the disclosure are plainly in fair use and are provided solely for the purposes of illustration of the invention in view of an urgent need to treat subjects as further discussed in detail below.

### DETAILED DESCRIPTION OF THE INVENTION

The subject innovation is now described in some instances, when necessary, with reference to the drawings. In the following description, for purposes of explanation, numerous specific details are set forth in order to provide a thorough understanding of the present invention. It may be evident, however, that the present invention may be practiced without these specific details. In other instances, well-known structures, methods, and devices are shown in block diagram form or with illustrations in order to facilitate describing the present invention. It is to be appreciated that certain aspects, modes, embodiments, variations and features of the invention are described below in various levels of detail in order to provide a substantial understanding of the present invention.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail. In general, chemical terminology is found in the International Union of Pure and Applied Chemistry GoldBook.² This disclosure is purposefully in plain words, known to a person of skill in the art, but Merriam-Webster's Online Dictionary is used, when appropriate, for terms not specifically demonstrated herein or not known in the art.³

As used in this specification and the appended claims, the singular forms “a,” “an” and “the” include plural referents unless the content clearly dictates otherwise. For example, reference to “a cell” includes a combination of two or more cells, and the like.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” means that other elements can also be present in addition to the defined elements presented. The use of “comprising” indicates inclusion rather than limitation. The term “including” can be interchanged with “comprising”.

The term “consisting of” refers to compositions, methods, and respective components thereof as described herein, which are exclusive of any element not recited in that description of the embodiment. The term “consisting of” can be used anywhere in the claims or otherwise in this entire disclosure to represent an exclusive feature that is stated in a claim or otherwise; and the term “consisting of” can be followed by the term “comprising”. For example, a method can consist of administering a composition consisting of Formula 1, Formula 2, Formula 3, or Formula 4 and an excipient; wherein the excipient comprises 3 ingredients. In this example, the 3 ingredients can be later stated, for example, in a dependent claim or in a statement of various suitable options for the 3 ingredients.

As used herein the term “consisting essentially of” refers to those elements required for a given embodiment. The term permits the presence of additional elements that do not materially affect the basic and novel or functional characteristic(s) of that embodiment of the invention. For example, a pharmaceutical formulation can consist essentially of yohimbine, meaning that a variety of excipients or other additives can be present in the formulation, but no other active pharmaceutical ingredient (API) is present in the formulation, except in formulations wherein an intended synergistic effect is demonstrated by the claims or examples herein (e.g., a formulation consisting essentially of yohimbine and Naloxone, or pharmaceutically acceptable salts thereof). In another example, a pharmaceutical formulation can consist essentially of yohimbine, meaning that the formulation is provided in the form of a nasal spray, an inhaled formulation, an orally administered formulation, or an injection formulation, each of which is tailored for a fast-acting agent, therapeutic agent, or antidote but not tailored for long-term administration (e.g., as a dietary supplement). In another example, in the case of a preventative supplement to purposefully prevent an overdose, the opposite, long-term supplementation, can be referred to with “consisting essentially of”. This example could be applied to a fentanyl addict who habitually purchases street fentanyl that is likely to be laced with xylazine. The term “consisting essentially of” can also be exemplified by plain language provided in the claims.

The term “statistically significant” or “significantly” refers to statistical significance and generally means a two-standard deviation (2SD) or greater difference.

As used herein, the term “subject” refers to a mammal, including but not limited to a dog, cat, horse, cow, pig, sheep, goat, rodent, or primate. Subjects can be house pets (e.g., dogs, cats), agricultural stock animals (e.g., cows, horses, pigs, chickens, etc.), laboratory animals (e.g., mice, rats, rabbits, etc.), but are not so limited. Subjects particularly include human subjects as described herein. The human subject may be a pediatric, adult, or a geriatric subject. The human subject may be of either sex.

As used herein, the terms “effective amount”, “therapeutically effective amount”, and “pharmaceutically effective amount” include an amount sufficient to prevent or ameliorate a manifestation of or a suspected manifestation of a medical condition, such as a drug overdose. The manifestation can be a sign or symptom or otherwise. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the medical condition and on the characteristics of the individual, such as general health, age, sex, body weight and tolerance to drugs. It will also depend on the degree, severity, and type of medical condition. The skilled artisan will be able to determine appropriate dosages depending on these and other factors. Examples of other factors can be route of administration and length of administration(s). The compositions can also be administered in combination with one or more additional therapeutic compounds.

The term “protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed.⁴ and in Harrison, et al., Compendium of Synthetic Organic Methods, Vols. 1-8.⁵ Examples of representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Examples of representative hydroxylprotecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample. For example, a compound that prevents epilepsy may reduce the frequency of seizures and/or reduce the severity of seizures.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The phrases “conjoint administration” and “administered conjointly” refer to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered at the same time, within one minute, 2 minutes, 4 minutes, 6 minutes, 10 minutes, 30 minutes, or an hour or 90 minutes of one another. Thus, an individual who receives such treatment can benefit from a combined effect of different therapeutic compounds.

As used herein, the terms “treat,” “treatment,” “treating,” or “amelioration” when used in reference to a disease, disorder, or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), sign(s), diminishment of extent of the deficit, stabilized (i.e., not worsening) state of a symptom or condition, delay or slowing of onset of symptoms or indications, and an increased lifespan as compared to that expected in the absence of treatment.

As used herein, the term “long-term” administration means that the therapeutic agent or drug is administered for a period of at least 12 weeks. This includes that the therapeutic agent or drug is administered such that it is effective over, or for, a period of at least 12 weeks and does not necessarily imply that the administration itself takes place for 12 weeks, e.g., if sustained release compositions or long-acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more.

The administration of the compositions contemplated herein may be carried out in any convenient manner, including by aerosol inhalation, injection, ingestion, transfusion, implantation, application (e.g., topical, otic, or ocular), or transplantation. Administration can be accomplished by an implant. In some embodiments, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into an artery, vein, lymph node, or organ (e.g., heart).

The terms: “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The term “prodrug” is intended to encompass compounds which, under physiologic conditions, are converted into the therapeutically active agents of the present invention (e.g., a compound selected from Formula 1, 2, 3, and/or 4). A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds selected from Formula 1, 2, 3, and/or 4 (in a non-limiting example) in a formulation represented above can be replaced with the corresponding suitable prodrug, e.g., wherein a hydroxyl in the parent compound is presented as an ester or a carbonate or carboxylic acid present in the parent compound is presented as an ester. In some embodiments, a “prodrug” is made by using an absorbing particle that subsequently releases an active form after administration.

In some embodiments, the decrease in the one or more signs or symptoms is evaluated according to the DSM-5.6 In some embodiments, signs are observed or measured by a health care provider. Symptoms can be reported by the subject. In some embodiments, the decrease of signs or symptoms occurs in less than about 120 minutes, 90 minutes, less than about 60 minutes, less than about 30 minutes, less than about 15 minutes, less than about 10 minutes, or less than about 5 minutes, or less than about 3 minutes, or less than about 1 minute.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “substance” consumed by a subject and suspected to be or involved in an overdose can be a small molecule less than 1000 MW or a large molecule not less than 1000 MW including biologics, oligonucleotides, peptides, oligosaccharides, and psychoactive large molecules. Any of the antagonists or therapeutic agents disclosed herein can be used as or in combination with small molecules and/or large molecules as discussed herein.

A subject can be one who has been previously diagnosed with or identified as suffering from or having a condition in need of treatment (e.g., an addiction or related behavior) or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. For example, a subject can be one who exhibits one or more risk factors for a condition, or one or more complications related to a condition or a subject who does not exhibit risk factors. A “subject in need” of treatment for a particular condition can be a subject having that condition, diagnosed as having that condition, or at risk of developing that condition.

### Pharmaceutical Compositions

The compositions and methods of the present invention may be utilized to prevent a need for other treatment, to provide benefit when other treatment(s) fail, or to treat an individual in need thereof. In some embodiments, the individual is suspected of needing treatment. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In some embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues, or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible compositions employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. Inhalation can include inhalation of a liquid (droplets or aerosol). Inhalation can include a micronized powder adhered to carrier particles or can be without carrier particles. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient(s) that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules, including sprinkle capsules and gelatin capsules), tablets, pills, dragées, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropyl methyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragées, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropyl methyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymers and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, micro-emulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art. See, e.g., Isselbacher, et al., (1996).⁷

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In other embodiments, the active compound will be administered once daily.

The subject or patient receiving this treatment is any animal in need, including primates, in particular humans; and other mammals such as equines bovine, porcine, sheep, feline, and canine; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino) ethanol, ethanolamine, ethylenediamine, N-methylglucamine, hydrabamine, 1H-imidazole, lithium, L-lysine, magnesium, 4-(2-hydroxyethyl) morpholine, piperazine, potassium, 1-(2-hydroxyethyl) pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, l-ascorbic acid, I-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, I-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, I-pyroglutamic acid, salicylic acid, sebacic acid, stearic acid, succinic acid, sulfuric acid, I-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent. The invention contemplates polymorphs, cocrystals, and amorphous forms of all substances discussed herein. As discussed above, solvates and/or hydrates can be formed by, for example, a slow evaporation whereby water and/or solvent remain hydrogen bonded with OH groups in the molecule. The formation of a solvate/hydrate can be quickly confirmed after the evaporation by using attenuated total reflectance Fourier transform infra-red spectroscopy wherein the solid solvate/hydrate is directly placed on the instrument and the subsequent IR spectrum is compared to the IR spectrum of the solid non-solvate, non-hydrate.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology and molecular biology can be found in The Merck Manual of Diagnosis and Therapy;⁸ The Encyclopedia of Molecular Cell Biology and Molecular Medicine;⁹ Molecular Biology and Biotechnology: a Comprehensive Desk Reference;¹⁰ Immunology;¹¹ Janeway's Immunobiology;¹² Lewin's Genes XI;¹³ Molecular Cloning: A Laboratory Manual.;¹⁴ Basic Methods in Molecular Biology;¹⁵ Laboratory Methods in Enzymology;¹⁶ Current Protocols in Molecular Biology (CPMB)¹⁷; Current Protocols in Protein Science (CPPS);¹⁸ and Current Protocols in Immunology (CPI).¹⁹

In the embodiments discussed and in any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

### Identification of a New Potent Inhibitor of Mycobacterial Growth

Amongst the infectious diseases, tuberculosis is the second only to SARS-COV-2 in terms of its lethality worldwide, with over one million victims in 2022, and ten times that number infected.¹ Although effective treatments for TB exist, the dosing regimens are long and complex. With the emergence of multi-drug resistant TB (MDR-TB), there is an urgent need for new anti-TB therapeutics.²⁰ Identifying new biomolecular targets in bacteria along with molecules capable of binding these targets can provide new options for treating drug-resistant infections.

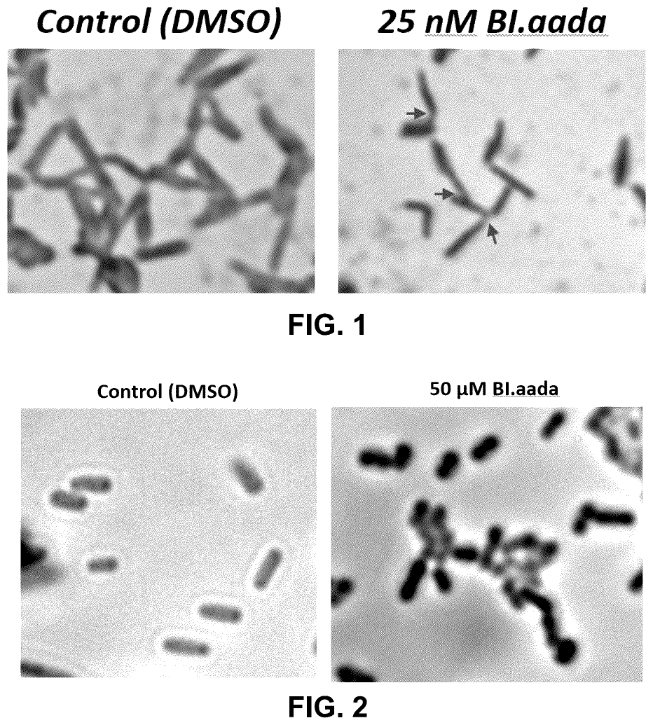

In an example (FIG. 1), we have now found that BI.aada shows nanomolar activities against several Mycobacteria—M smegmatis MIC 50 nM; M absescus MIC <500 nm; M avium <500 nM.

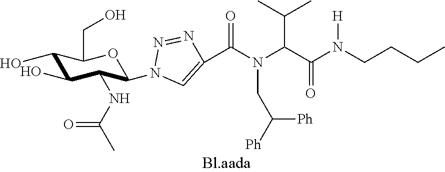

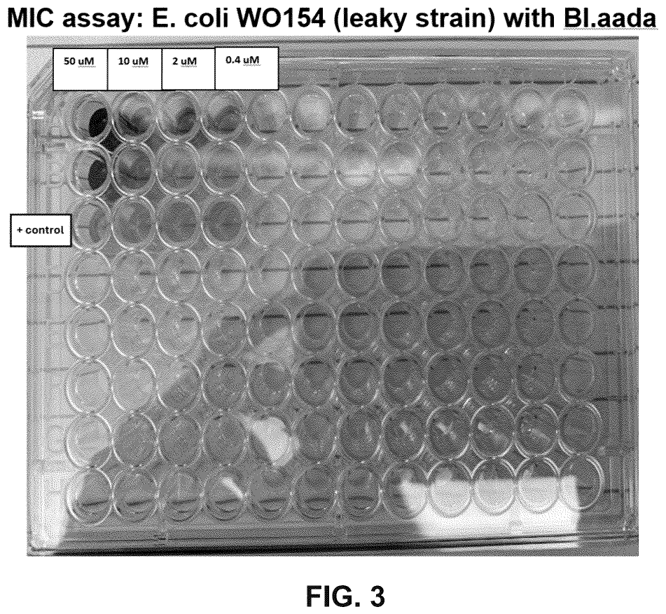

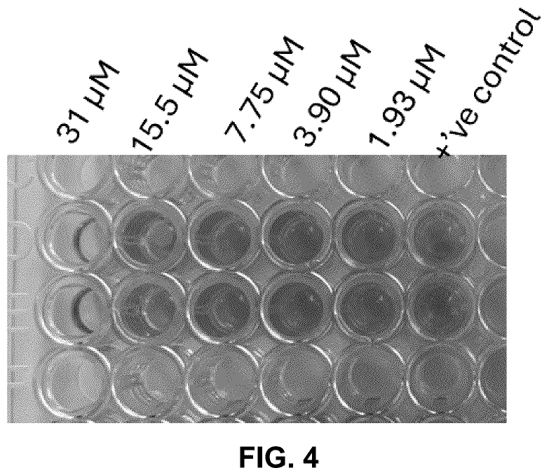

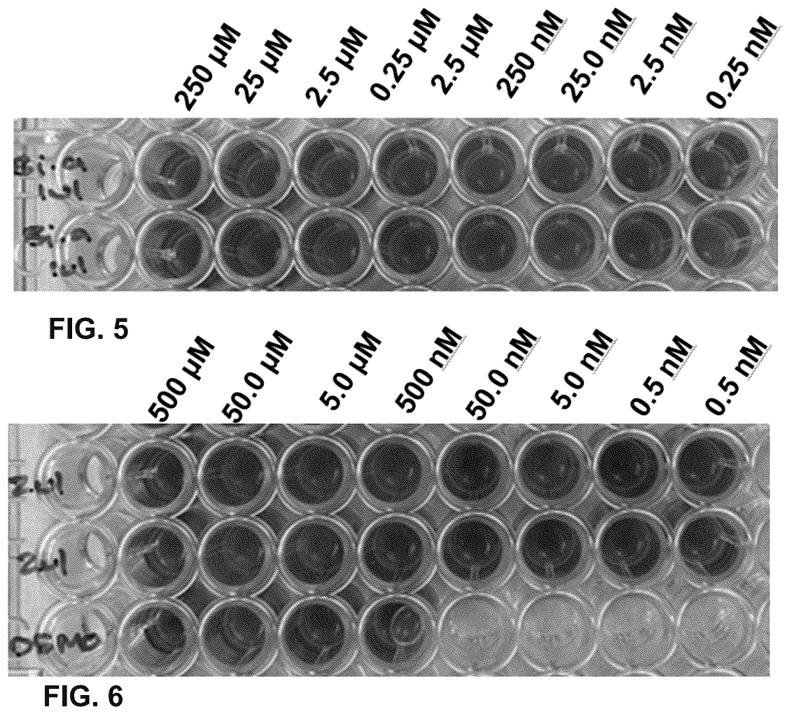

In FIG. 1, 25 nM=0.5×MIC. Arrows indicate aberrant division occurring along side wall (similar to that seen with B. subtilis+masarimycin). Magnification is 1000×. The compound BI.aada also has an MIC of 70 μM against the gram-negative E. coli WO0153, which has a leaky outer membrane, and no activity against E. coli K12, which has a normal membrane. Microscopy of both M. smegmatis and E. coli WO0153 in the presence of BI.aada exhibit altered cell morphology, consistent with action against a cell-wall target (FIG. 2, FIG. 3, FIG. 4, FIG. 5, and FIG. 6). In FIG. 2, sub-MIC treated E. coli WO153 demonstrates clumping phenotype compared to control. Also shows a “stubby” phenotype with incomplete daughter cell separation. The stubby phenotype is similar to that observed with NAG-thiazoline (lytic transglycosylase inhibitor) with E. coli. In FIG. 3, purple: no growth; pink: bacterial growth; and MIC determined as lowest concentration that inhibits growth: 50 μM. In FIG. 4, FIG. 5, and FIG. 6, purple: no growth; pink: bacterial growth; and MIC determined as lowest concentration that completely inhibits growth: Range 5-0.5 nM over multiple replicates.

N-Acetylglucosaminidases (GlcNAcases) play an important role in the remodeling and recycling of bacterial peptidoglycan. Inhibitors of bacterial GlcNAcases can serve as antibacterial agents and provide an opportunity for the development of new antibiotics. We report the synthesis of triazole derivatives of N-acetylglucosamine using a copper promoted azide-alkyne coupling reaction between 1-azido-N-acetyl-glucosamine and a small library of terminal alkynes prepared via the Ugi reaction. These compounds were evaluated for their ability to inhibit the growth of bacteria. Two compounds that show bacteriostatic activity against Bacillus were identified, with MIC values of approximately 60 μM in both cases. Bacillus subtilis cultured in the presence of sub-MIC amounts of the glycosyl triazole inhibitors exhibit an elongated phenotype characteristic of impaired cell division. This represents the first report of inhibitors of bacterial cell wall GlcNAcases that demonstrate inhibition of cell growth in whole cell assays.

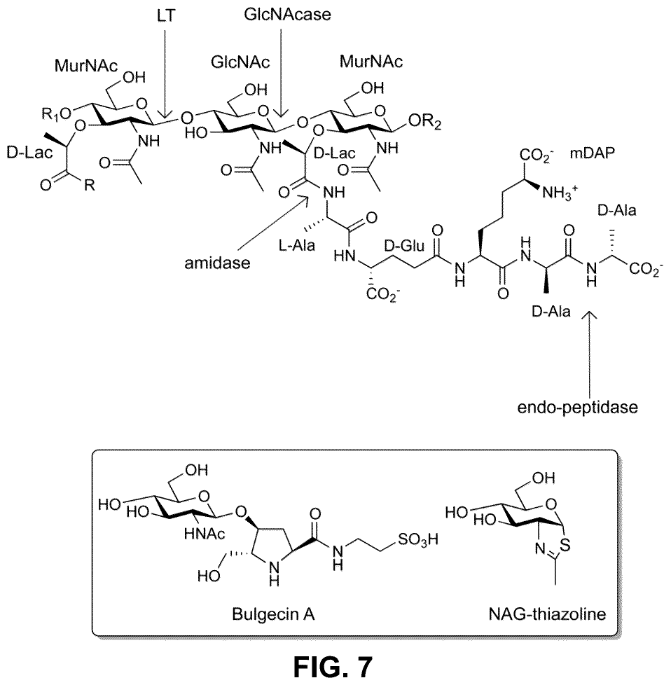

The microbial glycome contains numerous attractive targets for antibiotic discovery.²¹ Peptidoglycan (PG), the mesh-like heteropolymer that surrounds all bacterial cells (with the exception of mycoplasma), confers strength, support, and shape to bacteria, as well as providing resistance to internal turgor pressure.²² Maintaining the integrity of PG is essential to bacterial viability, which is reflected by the number of different classes of clinically important antibiotics that target its biosynthesis. The PG sacculus is composed of the amino sugars N-acetylglucosamine (GlcNAc) and N-acetylmuramic acid (MurNAc), linked via a β-(1,4)-glycosidic linkage to form an alternating copolymer.²³ Attached to the C-3 lactyl moiety of MurNAc is a highly variable pentapeptide composed of alternating L- and D-amino acids (FIG. 7).

FIG. 7 shows repeat structure of peptidoglycan (A1-γ chemotype) and cleavage sites of major bacterial autolysins; the box shows structures of bulgecin A and NAG-thiazoline, inhibitors of cell wall glycosyl hydrolases.

Adjacent strands of the glycan copolymer are inter-connected through cross-links between these peptides. The biosynthesis of PG, particularly the cytoplasmic assembly and periplasmic cross-linking steps, is fairly well understood. Some of the most successful antibiotics to date, including the β-lactams and vancomycin, inhibit enzymes involved in PG biosynthesis. Conversely, remodeling of the invariant glycan backbone of PG by glycosidases and lyases is poorly understood despite the important roles of these enzymes in cell growth and division.^{21,24} Many current cell wall acting antibiotics, which act on the highly variable stem peptide, are plagued by resistance issues. Because of the invariant glycan backbone, enzymes that act on it are particularly attractive antibiotic targets.

Autolysins, also referred to as peptidoglycan hydrolases, are a family of enzymes that include glycosidases and peptidases and are important for cell wall remodeling (FIG. 7).^{22,24,25} These enzymes play particularly important roles in cell division, motility and macromolecular assembly (e.g., pili). There are two classes of autolysins that act on the glycan backbone of PG—lytic transglycosylases (LT) and N-acetyl-glucosaminidases (GlcNAcase). GlcNAcases can be further categorized as endo-glycosidases, such as LytD from Bacillus subtilis, or exo-glycosidases like LytG.^{23,26,27} In Gram-positive bacteria, genetic and phenotypic analysis has identified an important role for GlcNAcases in vegetative cell growth and division. 28,29 Additionally, bacteria possess a cytosolic exo-acting GlcNAcase (e.g. NagZ) that acts on a disaccharide glycopeptide intermediate generated during PG recycling.^{30,31} Attempts to inhibit cleavage of the PG glycan backbone have focused on LTs from Gram-negative bacteria, which can be inhibited by the substrate analog NAG thiazoline and the natural product bulgecin A (FIG. 7).³²⁻³⁵ While these inhibitors show activity in vitro, they do not demonstrate a measurable minimum inhibitory concentration in whole cell assays. NAG thiazoline treatment of E. coli resulted in the formation of shorter cells without affecting cell viability.³⁵ NAG thiazoline has also been shown to inhibit a number of Gram-positive enzymes that are involved in N-glycan modification.^{36,37} Treatment of Pseudomonas aeruginosa with a NagZ inhibitor attenuated β-lactam resistance, demonstrating the potential for modulating antibiotic activity by inhibiting PG recycling.³⁸ An iminosugar based inhibitor of NagZ has recently been reported.³⁹

To date, no inhibitors targeting GlcNAcases that act on the cell wall have demonstrated antimicrobial activity in susceptibility tests. We have previously reported the synthesis of galactosyl and glucosyl triazoles as inhibitors for galactosidases and glucosidases.⁴⁰ Numerous subsequent studies have demonstrated the generality of the glycosyl triazole pharmacophore for glycosidase inhibition.⁴¹⁻⁴⁶ Based on this precedent, we sought to examine whether glycosyl triazole derivatives of GlcNAc would inhibit bacterial GlcNAcase activity. GlcNAc triazoles (GNTs) have previously been shown to inhibit O-GlcNAcase and have been examined as inhibitors for human hexosaminidase, but to the best of our knowledge have not been examined for anti-bacterial effects.^{47,48}

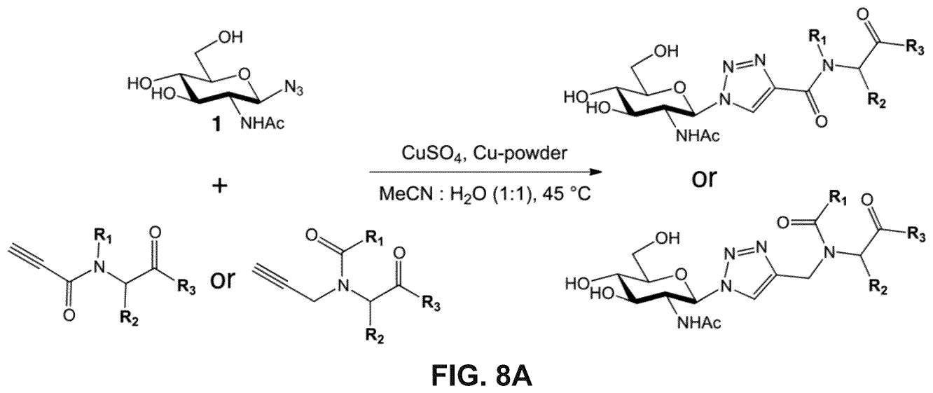

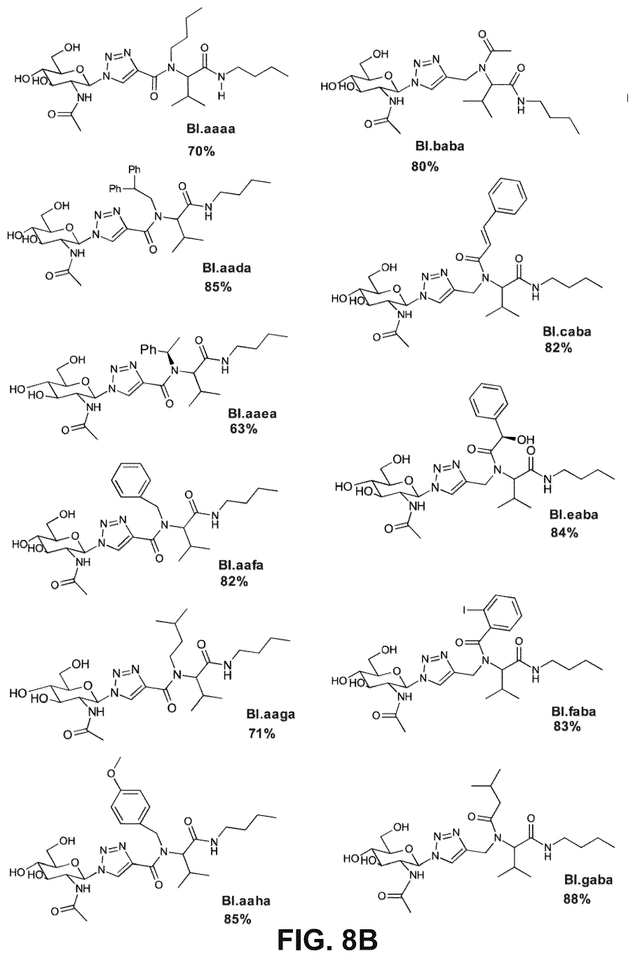

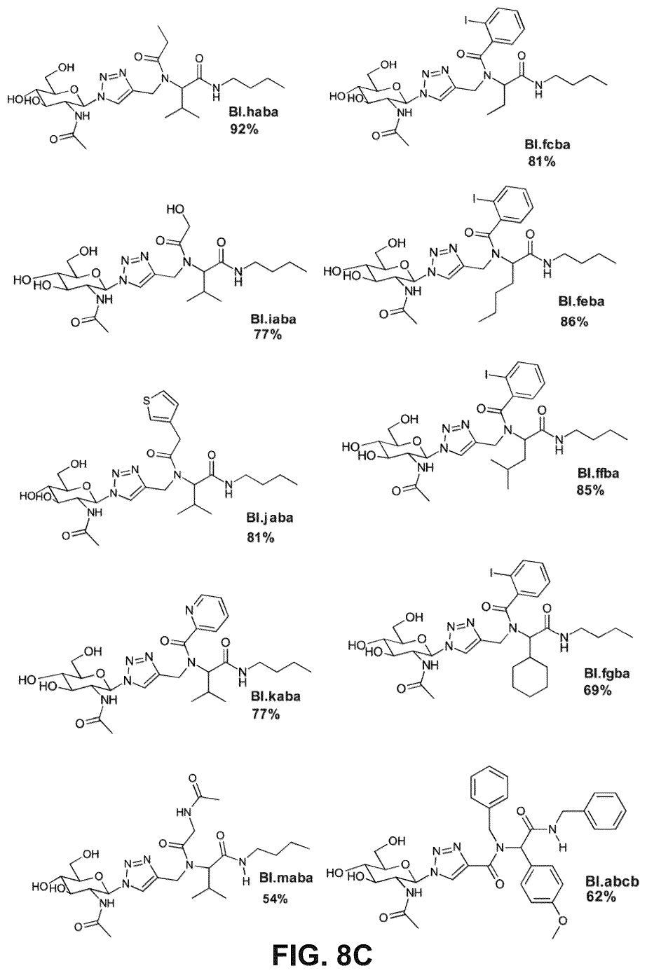

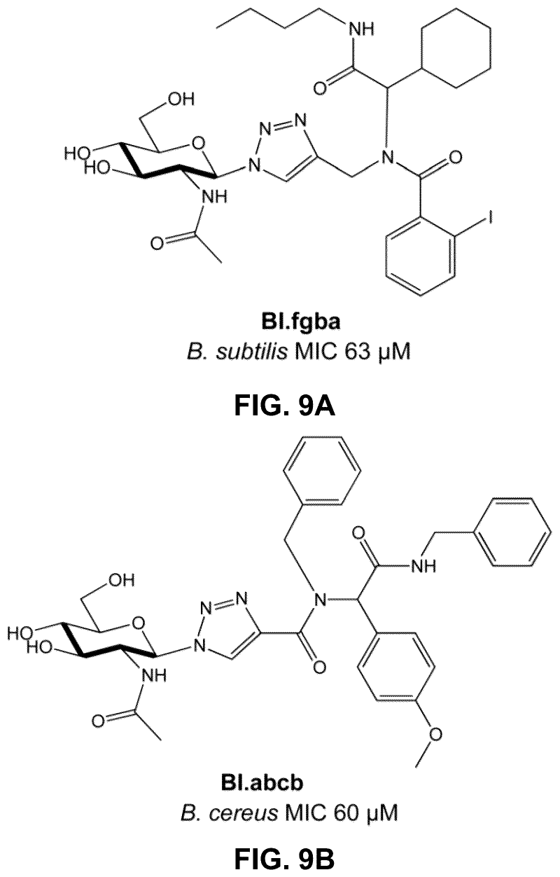

Results: We prepared a 21-member GNT library by coupling 1-azido-N-acetylglucosamine (1) to a series of terminal alkynes that were prepared using a multicomponent Ugi reaction (FIG. 8A, FIG. 8B, FIG. 8C).

Each Ugi reaction was carried out using either propargyl amine or propiolic acid as the alkyne component to provide either propionamides or N-propargyl amides as the diamide products. While Ugi-reactions can sometimes require prolonged reaction times, we identified conditions that reduced the reaction time to 4-5 hours at elevated temperature and in many cases proceeded to completion, enabling use of the Ugi product directly in the next reaction.⁴⁹⁻⁵¹ The Ugi-derived terminal alkynes were then subjected to a copper accelerated azide-alkyne coupling reaction using a redox couple of copper powder and copper (II) sulfate. To further facilitate synthesis of the library we simplified the purification of the compounds by using the alkyne building blocks in excess for the copper accelerated azide-alkyne coupling reaction. This ensured complete consumption of the azido sugar 1, and the unreacted alkyne residue could be easily removed using a silica plug after the reaction, eliminating the need for column chromatography. Removal of trace copper salts from the reaction was carried out by incubating the reaction solution with a commercially available copper chelating resin prior to purification using the silica plug. The library of GNTs was screened for its ability to inhibit the growth of a panel of different Gram-positive bacteria. Glycosyl triazole compounds were screened in a whole cell assay against a number of Gram positive organisms using a resazurin microtiter assay.⁵²

The library was initially screened against all test organisms at a single concentration (250 μM) (ESIt). This high initial concentration was chosen as many soluble derivatives of PG bind to bacterial autolysins with K_{D} values in the high micromolar range.⁵³ Compounds showing at least 40% inhibition in growth were selected for further investigation. Follow up studies with 6 compounds that inhibited growth by more than 40% indicated that not all of these compounds reduced bacterial growth in a concentration dependent manner. We identified two compounds that inhibited the growth of Bacillus cereus and Bacillus subtilis in the micromolar range (FIG. 9). GNT BI.abcb inhibited B. cereus with an MIC value of 39 mg mL⁻¹ (60 μM), and BI.fgba inhibited B. subtilis with an MIC value of 45 mg mL⁻¹ (63 μM). Both BI.abcb and BI.fgba were screened in a standard bactericidal assay.⁵⁴ Briefly, B. subtilis and B. cereus were grown in either the absence or presence of inhibitor (at the MIC) for 4 hours.

The cells were harvested, washed and subject to serial dilution prior to plating on nutrient broth agar. Colony counts for growth in the presence of BI.fgba or BI.abcb (8.50±3.00×10⁷ cfu mL⁻¹ and 7.00±1.70×10⁸ cfu mL⁻¹ respectively) were similar to controls in the absence of the compounds (9.40±2.88×107 cfu mL⁻¹ for B. subtilis, and 2.67±1.53×108 cfu mL⁻¹ for B. cereus) indicating that these compounds are bacteriostatic in nature. In order to assess the role of the glycone and aglycone components in inhibition, the galactose derivatives of BI.fgba and BI.abcb were synthesized and tested, and exhibited no antimicrobial activity.

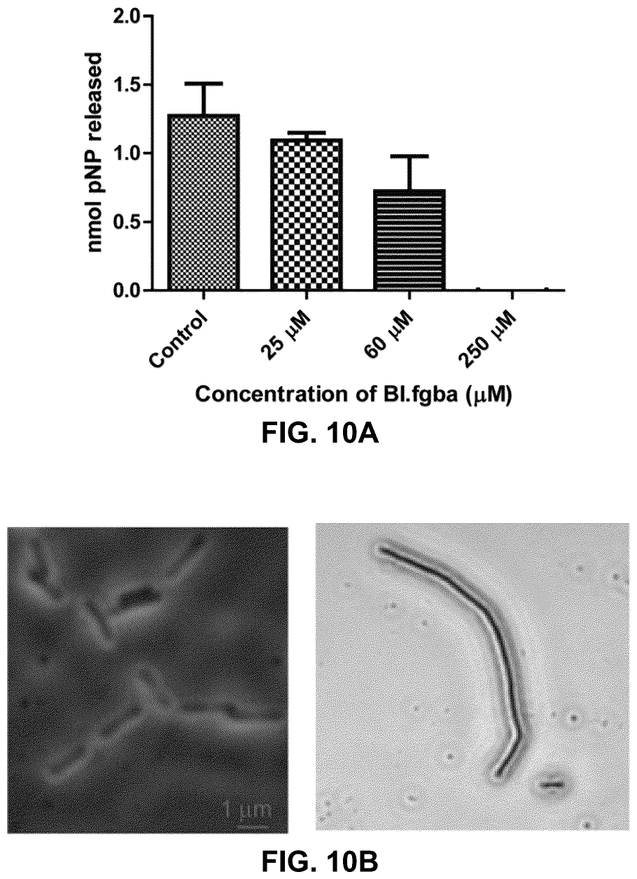

Incubation of B. subtilis cells with the synthetic substrate β-p-nitrophenyl GlcNAc (pNP-GlcNAc) in the presence of BI.fgba resulted in a concentration dependent inhibition of nitrophenol release (FIG. 10A), with complete inhibition observed at 250 μM inhibitor. This result confirms that the likely bacterial target is indeed a GlcNAcase. The differences in potency between the in vitro assay and the anti-bacterial assay may reflect poor access to the target in the whole cell assay. Additionally, it should be noted that pNP-GlcNAc is not the natural substrate for a bacterial GlcNAcase, and lacks many protein-substrate contacts such as the stem peptide as well as an extended glycan chain. Thus, the ability of BI.fgba to inhibit pNP-GlcNAc hydrolysis does not necessarily reflect its ability to inhibit peptidoglycan hydrolysis. Treatment of B. subtilis with BI.fgba at a concentration below its MIC (0.8×MIC), resulted in a phenotype with highly elongated cells and the appearance of chains of cells (FIG. 10B).

FIG. 10A shows dose dependent inhibition of pNP-GlcNAc hydrolysis by intact B. subtilis cells in the presence of BI.fgba confirming that the target is a GlcNAcase. FIG. 10B shows morphological changes to B. subtilis upon exposure to BI.fgba. Control cells (left) were grown in the presence of 1% DMSO and show the typical rod-shaped cells. Cells on the right were treated with 0.8×MIC (48 UM) BI.fgba and exhibit an elongated phenotype.

This is suggestive of a disruption in the cell division/septation machinery, processes that are known to require GlcNAcase activity. This phenotype is reminiscent of that observed in Lactococcus lactis that lacks AcmA or AcmD, which are orthologs of LytG in B. subtilis and involved in cleavage of the septum during cell division.^{55,56} In summary we have identified two inhibitors of bacterial GlcNAcases based on a glycosyl triazole scaffold. The bacteriostatic activity of both of these GNTs, in conjunction with biochemical evidence for inhibition for pNP-GlcNAc hydrolysis and the impaired cell division phenotype of cells treated with BI.fgba, is strongly suggestive of disrupted autolysin activity. There have been prior reports of compounds that inhibit purified bacterial GlcNAcases in vitro, as well as compounds that are known GlcNAcase inhibitors that exhibit antibacterial activity indirectly by sensitizing the bacteria to β-lactam antibiotics. However, to the best of our knowledge, this is the first report of compounds that exhibit inhibition of GlcNAcase activity that also directly reduce bacterial growth. While the MIC values exhibited by BI.fgba and BI.abcb are modest, our results demonstrate an important proof of concept and validate the glycosyl triazole scaffold as a viable one for further optimization and development. Efforts have been directed at identifying the molecular target(s) of these compounds and improving the potency of the GNTs.

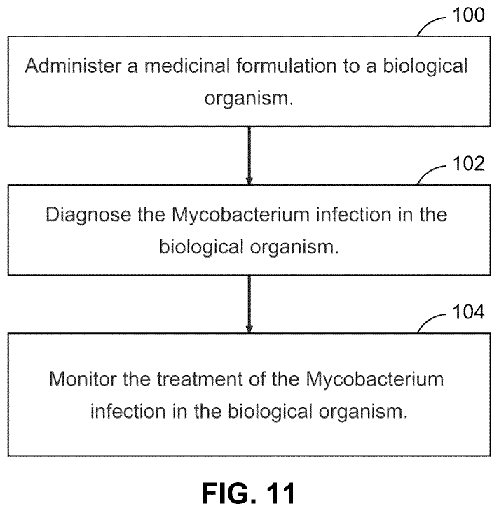

FIG. 11, is a flow diagram illustrating example steps of addressing a Mycobacterium infection. In step 100, the method involves administering a medicinal formulation to the biological organism. This medicinal formulation is designed for treating a Mycobacterium infection, including a tuberculosis infection, by administering a therapeutically effective amount of a small molecule.

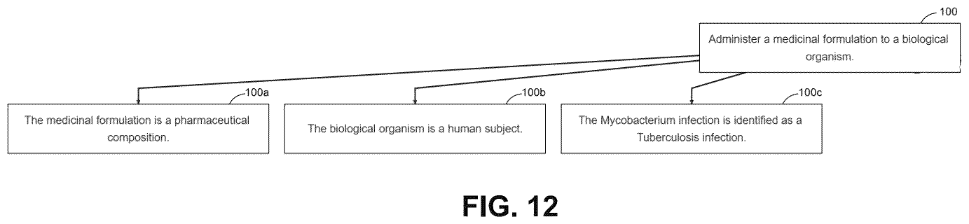

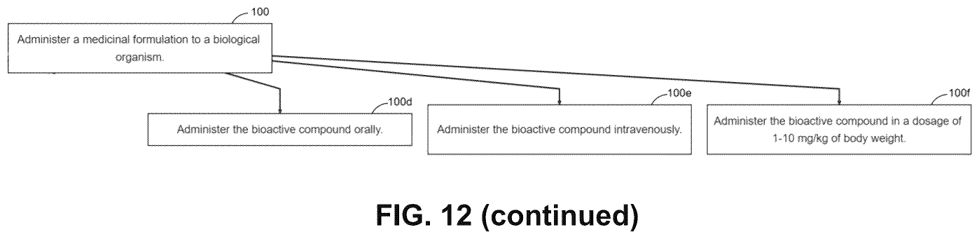

The small molecule comprises N-acetylglucosamine or N-glycolylglucosamine and a triazole or any molecule disclosed herein, which interact with the Mycobacterium to provide the chemical structure for the therapeutic effect. Sub-step 100-a (FIG. 12) specifies that the medicinal formulation is a pharmaceutical composition. Administering the small molecule in the form of a pharmaceutical composition facilitates the delivery of the therapeutic agent to the human subject. The pharmaceutical composition ensures that the small molecule, which includes N-acetylglucosamine or N-glycolylglucosamine and a triazole, is administered in a form that is effective for treatment. Sub-step 100-b (FIG. 12) indicates that the biological organism is a human subject. The procedure outlines the approach for treating a human subject with a Mycobacterium infection. Sub-step 100-c clarifies that the Mycobacterium infection being treated is a tuberculosis infection.

Administering a therapeutically effective amount of a small molecule aims to treat Mycobacterium infection, including tuberculosis. This step ensures that the treatment is aimed at combating tuberculosis, a type of Mycobacterium infection. Sub-step 100-d describes that the bioactive compound is administered orally. Administering the small molecule in the form of a pharmaceutical composition facilitates the delivery of the therapeutic agent to the human subject. Additionally, the small molecule comprising N-acetylglucosamine or N-glycolylglucosamine and a triazole provides the chemical structure to treat the Mycobacterium infection. Sub-step 100-e (FIG. 12) mentions that the bioactive compound can also be administered intravenously. Similar to oral administration, administering the small molecule in the form of a pharmaceutical composition facilitates the delivery of the therapeutic agent to the human subject. The small molecule's composition remains the same, ensuring its effectiveness in treating the Mycobacterium infection. Sub-step 100-f specifies that the bioactive compound is administered in a dosage of 1-10 mg/kg of body weight. This dosage range ensures that the small molecule is administered in a therapeutically effective amount. Administering the small molecule in the form of a pharmaceutical composition facilitates the delivery of the therapeutic agent to the human subject, and the specified dosage ensures that the treatment is both safe and effective.

In sub-step 100-a, the medicinal formulation is specified to be a pharmaceutical composition. This sub-step involves the medicinal formulation and pharmaceutical composition.

The medicinal formulation is defined as a pharmaceutical composition, which is an aspect of the treatment method for Mycobacterium infection, including tuberculosis infection. The medicinal formulation, as described, is administered to treat a Mycobacterium infection in a biological organism. The medicinal formulation comprises a bioactive compound that includes N-acetylglucosamine or N-glycolylglucosamine and triazole. The bioactive compound interacts with the Mycobacterium, providing the chemical structure for therapeutic effect. Administering the small molecule in the form of a pharmaceutical composition facilitates the delivery of the therapeutic agent to the human subject. The method for treating a Mycobacterium infection, including a tuberculosis infection, involves administering a therapeutically effective amount of a small molecule. This small molecule comprises N-acetylglucosamine or N-glycolylglucosamine and a triazole and is administered in the form of a pharmaceutical composition.

The pharmaceutical composition is designed to deliver the dosage for treatment and to alleviate the symptoms of Mycobacterium infection, including tuberculosis. The pharmaceutical composition, as part of the medicinal formulation, ensures that the small molecule is effectively delivered to the human subject. This composition is for the treatment method, as it provides a stable and effective means of administering the bioactive compound. The small molecule's inclusion of N-acetylglucosamine or N-glycolylglucosamine and triazole is for its therapeutic effect, targeting the Mycobacterium infection. In summary, sub-step 100-a highlights the medicinal formulation being a pharmaceutical composition. This ensures the effective delivery of the small molecule, which comprises N-acetylglucosamine or N-glycolylglucosamine and triazole, to treat Mycobacterium infection, including tuberculosis, in a human subject. The pharmaceutical composition facilitates the administration of a therapeutically effective amount of the small molecule, providing the chemical structure for the treatment. In step 100-b, the focus is on the biological organism being a human subject.

The method for treating a Mycobacterium infection, specifically a tuberculosis infection, involves administering a medicinal formulation to a human subject. This medicinal formulation is a pharmaceutical composition that includes a bioactive compound comprising N-acetylglucosamine or N-glycolylglucosamine and triazole. This procedure outlines the approach for treating a human subject with a Mycobacterium infection. The method of treating a human subject involves administering a therapeutically effective amount of a small molecule. This small molecule, which includes N-acetylglucosamine or N-glycolylglucosamine and triazole, is designed to provide the chemical structure to treat the Mycobacterium infection. The small molecule is administered in the form of a pharmaceutical composition to facilitate the delivery of the therapeutic agent to the human subject. Administering the small molecule in this form ensures that the therapeutic agent is effectively delivered to the human subject, thereby alleviating the symptoms of the Mycobacterium infection, including tuberculosis.

The procedure for treating a human subject with a Mycobacterium infection, including tuberculosis, by administering a therapeutically effective amount of a small molecule, is an aspect of the method. This method ensures that the human subject receives the appropriate treatment to combat the infection effectively.

In step 100-c (FIG. 12), the focus is on specifying that the Mycobacterium infection being treated is a tuberculosis infection. This step narrows down the type of Mycobacterium infection, ensuring that the treatment method is tailored for tuberculosis. The method for treating a Mycobacterium infection, including a tuberculosis infection, involves administering a therapeutically effective amount of a small molecule. This small molecule comprises N-acetylglucosamine or N-glycolylglucosamine and a triazole or any small molecule disclosed herein, which are for providing the chemical structure to treat the infection. The small molecule is administered in the form of a pharmaceutical composition, which facilitates the delivery of the therapeutic agent to the human subject. Administering a therapeutically effective amount of a small molecule aims to treat Mycobacterium infection, including tuberculosis. This is achieved by the small molecule interacting with the Mycobacterium, thereby alleviating the symptoms of the infection.

The method is designed to apply the treatment to a human subject, ensuring that the approach is suitable for treating a human patient with a Mycobacterium infection. In summary, step 100-c specifies that the Mycobacterium infection being treated is a tuberculosis infection, and the method involves administering a small molecule comprising N-acetylglucosamine or N-glycolylglucosamine and a triazole in the form of a pharmaceutical composition to a human subject. The purpose is to alleviate the symptoms of the tuberculosis infection by delivering the dosage of the small molecule to the patient.

In step 100-d (FIG. 12), the method involves administering the bioactive compound orally. The method for treating a Mycobacterium infection, including a tuberculosis infection, involves administering a therapeutically effective amount of a small molecule. This small molecule comprises N-acetylglucosamine or N-glycolylglucosamine and a triazole, which are for providing the chemical structure to treat the Mycobacterium infection. The medicinal formulation, which is a pharmaceutical composition, facilitates the delivery of the therapeutic agent to the human subject. Administering the medicinal formulation orally ensures that the bioactive compound is effectively delivered to the biological organism. Administering the small molecule in the form of a pharmaceutical composition facilitates the delivery of the therapeutic agent to the human subject. The small molecule, comprising N-acetylglucosamine or N-glycolylglucosamine and a triazole, is administered in the form of a pharmaceutical composition to provide the chemical structure to treat the Mycobacterium infection. The method of treating a human subject with a Mycobacterium infection, including a tuberculosis infection, by administering a therapeutically effective amount of a small molecule, is designed to alleviate the symptoms of the infection.

The small molecule is administered orally to ensure that the therapeutic agent is effectively absorbed and utilized by the body. This method outlines the approach for treating a human subject with a Mycobacterium infection, ensuring that the treatment is effective.

In step 100-e, the focus is on the administration of the bioactive compound intravenously. The method for treating a Mycobacterium infection, including a tuberculosis infection, involves administering a medicinal formulation to a biological organism. This medicinal formulation is a pharmaceutical composition that includes a bioactive compound comprising N-acetylglucosamine or N-glycolylglucosamine and triazole. The bioactive compound interacts with the Mycobacterium to treat the infection. The bioactive compound is administered intravenously, which is a route of administration. Administering the small molecule in the form of a pharmaceutical composition facilitates the delivery of the therapeutic agent to the human subject.

The small molecule comprising N-acetylglucosamine or N-glycolylglucosamine and a triazole provides the chemical structure to treat the Mycobacterium infection. This method is designed to deliver a therapeutically effective amount of the small molecule to alleviate the symptoms of the Mycobacterium infection, including tuberculosis. The intravenous administration ensures that the bioactive compound is delivered directly into the bloodstream, allowing for rapid and efficient distribution throughout the body. This route of administration is particularly useful for patients who may not be able to take oral medications or for situations where a rapid onset of action is required. In summary, step 100-e involves the intravenous administration of a bioactive compound, which is a small molecule comprising N-acetylglucosamine or N-glycolylglucosamine and triazole, as part of a pharmaceutical composition. This method is used to treat Mycobacterium infections, including tuberculosis, by delivering a therapeutically effective amount of the compound directly into the bloodstream for rapid and efficient treatment.

In step 100-f (FIG. 12), the method involves administering a bioactive compound in a dosage of 1-10 mg/kg of body weight. This step is part of a broader method for treating a Mycobacterium infection, specifically a tuberculosis infection, in a biological organism, which is typically a human subject.

The medicinal formulation used in this method is a pharmaceutical composition that includes a bioactive compound comprising N-acetylglucosamine or N-glycolylglucosamine and triazole. The bioactive compound is administered in a dosage of 1-10 mg/kg of body weight. This specific dosage range ensures the therapeutic efficacy of the treatment while minimizing potential side effects. Administering the small molecule in the form of a pharmaceutical composition facilitates the delivery of the therapeutic agent to the human subject. The small molecule comprising N-acetylglucosamine or N-glycolylglucosamine and a triazole provides the chemical structure to treat the Mycobacterium infection, including tuberculosis. The method of treating a human subject involves administering a therapeutically effective amount of this small molecule. The small molecule is administered in the form of a pharmaceutical composition, which ensures that the bioactive compound is delivered effectively to the site of infection. The pharmaceutical composition is designed to deliver the dosage for treatment and to alleviate the symptoms of the Mycobacterium infection, including tuberculosis. In summary, step 100-f specifies the administration of a bioactive compound in a dosage range of 1-10 mg/kg of body weight, which is for the effective treatment of Mycobacterium infections in human subjects. The small molecule, comprising N-acetylglucosamine or N-glycolylglucosamine and triazole, is delivered in the form of a pharmaceutical composition to ensure optimal therapeutic outcomes.

In step 102 (FIG. 11), the method further comprises diagnosing the Mycobacterium infection in the biological organism. This step involves identifying the presence of the Mycobacterium infection, which is for initiating the appropriate treatment regimen. The method for treating a Mycobacterium infection, including a tuberculosis infection, involves administering a therapeutically effective amount of a small molecule. This small molecule comprises N-acetylglucosamine or N-glycolylglucosamine and a triazole, which are for providing the chemical structure to treat the infection. The small molecule is administered in the form of a pharmaceutical composition to facilitate the delivery of the therapeutic agent to the human subject. Diagnosing the Mycobacterium infection ensures that the treatment is specifically targeted towards the infection, thereby increasing the efficacy of the therapeutic intervention. This diagnostic step is an additional step in the treatment process, ensuring that the presence of the infection is confirmed before proceeding with the administration of the medicinal formulation

In step 104 (FIG. 11), the method further comprises monitoring the treatment of the Mycobacterium infection in the biological organism. This step involves tracking the progress of the treatment to ensure its effectiveness and to make any necessary adjustments. The method for treating a Mycobacterium infection, including a tuberculosis infection, involves administering a therapeutically effective amount of a small molecule. This small molecule comprises N-acetylglucosamine or N-glycolylglucosamine and a triazole, which are for providing the chemical structure to treat the Mycobacterium infection. The small molecule is administered in the form of a pharmaceutical composition to facilitate the delivery of the therapeutic agent to the human subject. Monitoring the treatment tracks the progress and effectiveness of the administered small molecule in treating the Mycobacterium infection. This involves regular assessments and evaluations to determine if the infection is responding to the treatment and to identify any potential side effects or complications. Monitoring is a step in the treatment process as it ensures that the therapeutic approach is working as intended and allows for timely interventions if the treatment needs to be adjusted. Overall, step 104 emphasizes continuous monitoring in the treatment of Mycobacterium infections to achieve the desired therapeutic outcomes and to ensure the safety and well-being of the human subject undergoing treatment.

The method for treating a Mycobacterium infection, including a tuberculosis infection, involves administering a medicinal formulation to a biological organism, specifically a human subject. This medicinal formulation is a pharmaceutical composition that comprises a bioactive compound, which includes N-acetylglucosamine or N-glycolylglucosamine and triazole. The bioactive compound interacts with the Mycobacterium to treat the infection. The process begins with the method of treating a human subject, which involves administering a therapeutically effective amount of a small molecule. This small molecule is composed of N-acetylglucosamine or N-glycolylglucosamine and triazole, providing the chemical structure to treat the Mycobacterium infection. Administering this small molecule in the form of a pharmaceutical composition facilitates the delivery of the therapeutic agent to the human subject. The medicinal formulation, which is a pharmaceutical composition, is designed to treat Mycobacterium infections, including tuberculosis.

The bioactive compound within the formulation interacts with the Mycobacterium, thereby alleviating the symptoms of the infection. The method specifies that the biological organism being treated is a human subject, and the Mycobacterium infection being treated is specifically a tuberculosis infection. Additionally, the method includes steps for diagnosing the Mycobacterium infection in the biological organism and monitoring the treatment progress. The bioactive compound can be administered either orally or intravenously, depending on the specific requirements of the treatment. The dosage of the bioactive compound is specified to be in the range of 1-10 mg/kg of body weight, ensuring the delivery of a therapeutically effective amount to treat the infection. In summary, the method for treating a Mycobacterium infection involves a detailed procedure that includes diagnosing the infection, administering a pharmaceutical composition containing a bioactive compound (N-acetylglucosamine or N-glycolylglucosamine and triazole), and monitoring the treatment progress. The bioactive compound is administered in a specific dosage range and can be delivered orally or intravenously to ensure effective treatment of the infection in a human subject.

In any interpretation of the claims appended hereto, it is noted that no claims or claim elements are intended to invoke or be interpreted under 35 U.S.C. 112 (f) unless the words “means for” or “step for” are explicitly used in the particular claim.

In general, any combination of disclosed features, components and methods described herein is possible. Steps of a method can be performed in any order that is physically possible.

All cited references are incorporated by reference herein. Although embodiments have been disclosed, it is not desired to be limited thereby. Rather, the scope should be determined only by the appended claims.

While various embodiments of the present disclosure have been described in detail, it is apparent that modifications and alterations of those embodiments will occur to those skilled in the art. However, it is to be expressly understood that such modifications and alterations are within the scope and spirit of the present disclosure, as set forth in the following claims.

While contemplating (and disclosing to save lives) the technology herein, in a discussion, study or a reading of the details, features, embodiments, aspects, any figure or any part of any figure, and/or examples of the technology disclosed herein, any of the features, embodiments, aspects, and/or examples herein can be optionally inter-combined (or inter-discussed) with the example details (i.e., numbered details) listed below, and any portion (or aspect) of any detail below can be inter-combined with any portion (even the smallest detail) of any feature or example disclosed herein:

Detail 1: A method for treating and/or preventing a mycobacteria infection in a subject in need thereof, the method comprising: administering a therapeutically effective amount of a small molecule comprising an N-acetylglucosamine and a triazole to the subject; or administering a therapeutically effective amount of a small molecule comprising any of Formula 1, Formula 2, Formula 3, and/or Formula 4 to the subject.

Detail 2: The method of detail 1, wherein the small molecule comprises N-2,2-Diphenylethyl-N-[2-methyl-1-(N-butylcarbamoyl) propyl]-1-[(2R,3R,4R,5S,6R)-3-acetylamino-4,5-dihydroxy-6-(hydroxymethyl)tetrahydro-2H-pyran-2-yl]-1H-1,2,3-triazole-4-carboxamide; or N-2,2-diphenylethyl-N-[2-methyl-1-(N-butylcarbamoyl) propyl]-1-[(2R,3R,4R,5S,6R)-4,5-dihydroxy-6-(hydroxymethyl)-3-(methylcarbonylamino)tetrahydro-2H-pyran-2-yl]-1H-1,2,3-triazole-4-carboxamide to the subject; or administering a therapeutically effective amount of a chemical structure with different substituents; wherein the chemical structure comprises N-Methyl [({1-[(2R,3R,4R,5S,6R)-3-acetylamino-4,5-dihydroxy-6-(hydroxymethyl)tetrahydro-2H-pyran-2-yl]-1H-1,2,3-triazol-4-yl}methyl)amino]acetamide; or N-methyl-[{(1-[(2R,3R,4R,5S,6R)-4,5-dihydroxy-6-(hydroxymethyl)-3-(methylcarbonylamino)tetrahydro-2H-pyran-2-yl]-1H-1,2,3-triazol-4-yl}methyl)amino]ethanamide.

Detail 3: The method of detail 1 or 2, wherein any carbon atom in the small molecule is replaced by a boron atom; and wherein the boron atom is configured to overcome a drug-resistance of a mycobacteria; and/or wherein more than one carbon atom is replaced by a boron atom that overcomes a drug-resistance of a mycobacteria.

Detail 4: The method of detail 1 or 2, wherein the small molecule is administered in a dosage form selected from the group consisting of a tablet, a capsule, a solution, a suspension, an emulsion, a powder, a granule, a suppository, an injection, an inhalant, and a transdermal patch.

Detail 5: The method of detail 1 or 2, wherein the different substituents comprise: hydrogen, deuterium, halogen, alkyl, cycloalkyl, heteroalkyl, arylalkyl, alkoxy, aryloxy, amino, silyl, alkenyl, cycloalkenyl, heteroalkenyl, alkynyl, aryl, heteroaryl, acyl, carbonyl, carboxylic acid, ester, nitrile, isonitrile, sulfanyl, sulfinyl, sulfonyl, phosphino, boryl, and/or combinations thereof.

Detail 6: The method of detail 1 or 2, wherein the sustained release formulation comprises a biodegradable polymer matrix, a hydrogel, a liposome, a microsphere, a nanoparticle, or a combination thereof, and wherein the sustained release formulation provides a controlled release of the small molecule over a period of at least 24 hours, at least 48 hours, at least 72 hours, at least one week, at least two weeks, at least one month, at least two months, at least three months, at least six months, or at least one year.

Detail 7: The method of detail 1 or 2, wherein the one or more additional therapeutic agents are administered simultaneously, sequentially, or alternately with the small molecule, and wherein the one or more additional therapeutic agents are administered in a therapeutically effective amount to enhance the efficacy of the small molecule, to reduce the side effects of the small molecule, or to provide a synergistic effect with the small molecule.

Detail 8: The method of detail 7, wherein the antibiotics are selected from the group consisting of rifampin, isoniazid, pyrazinamide, ethambutol, streptomycin, rifabutin, rifapentine, moxifloxacin, levofloxacin, amikacin, capreomycin, ethionamide, cycloserine, para-aminosalicylic acid, and bedaquiline; wherein the antiviral agents are selected from the group consisting of efavirenz, nevirapine, delavirdine, zidovudine, lamivudine, abacavir, tenofovir, emtricitabine, rilpivirine, etravirine, and dolutegravir; wherein the antifungal agents are selected from the group consisting of fluconazole, itraconazole, voriconazole, posaconazole, amphotericin B, and caspofungin; wherein the anti-inflammatory agents are selected from the group consisting of corticosteroids, nonsteroidal anti-inflammatory drugs (NSAIDs), and aspirin; and wherein the immunomodulatory agents are selected from the group consisting of interferon-gamma, interleukin-2, interleukin-12, and tumor necrosis factor-alpha.

Detail 9: The method of detail 1 or 2, wherein the mycobacteria infection is a pulmonary infection, an extrapulmonary infection, or a disseminated infection; and wherein the extrapulmonary infection is selected from the group consisting of lymphadenitis, pleuritis, pericarditis, peritonitis, meningitis, encephalitis, osteomyelitis, arthritis, genitourinary tract infection, and cutaneous infection.

Detail 10: The method of detail 1 or 2, wherein the subject is an adult, a child, an infant, a neonate, an elderly person, a pregnant woman, a nursing mother, an immunocompromised person, a person with HIV/AIDS and/or COVID-19, a person with diabetes, a person with cancer, a person with a history of organ transplantation, a person with a history of substance abuse, a person with a history of incarceration, a person living in a long-term care facility, a healthcare worker, a laboratory worker, a traveler to an endemic area, or a person in close contact with an infected individual.

Detail 11: The method of detail 1 or 2, wherein the subject is immunocompromised due to a genetic disorder, an acquired disorder, a medical treatment, or a combination thereof; and wherein the genetic disorder is selected from the group consisting of severe combined immunodeficiency (SCID), chronic granulomatous disease (CGD), and complement deficiencies; wherein the acquired disorder is selected from the group consisting of HIV/AIDS and/or COVID-19, malnutrition, and immunosuppressive medications; and wherein the medical treatment is selected from the group consisting of chemotherapy, radiation therapy, and immunosuppressive therapy for organ transplantation or autoimmune diseases.

Detail 12: The method of detail 1 or 2, wherein the latent mycobacteria infection is characterized by the presence of mycobacteria in the subject without active disease symptoms, and wherein the latent mycobacteria infection is diagnosed by a positive tuberculin skin test or a positive interferon-gamma release assay in the absence of active disease symptoms.

Detail 13: The method of detail 1 or 2, wherein the active mycobacteria infection is characterized by the presence of mycobacteria in the subject with active disease symptoms, and wherein the active mycobacteria infection is diagnosed by a positive culture, a positive acid-fast stain, or a positive nucleic acid amplification test from a clinical specimen obtained from the subject.

Detail 14: The method of detail 1 or 2, wherein the small molecule is administered prophylactically to prevent a primary mycobacteria infection in a subject at risk of exposure to mycobacteria, to prevent the reactivation of a latent mycobacteria infection in a subject with a positive tuberculin skin test or a positive interferon-gamma release assay, or to prevent the recurrence of an active mycobacteria infection in a subject with a history of previous infection.

Detail 15: The method of detail 1 or 2, wherein the small molecule is administered therapeutically to eradicate or control the growth of mycobacteria in a subject with an active mycobacteria infection, and wherein the small molecule is administered as a primary therapy, as an adjunctive therapy to other anti-mycobacterial agents, or as a salvage therapy for drug-resistant mycobacteria infections.

Detail 16: The method of detail 1 or 2, wherein the bacterial load is measured by the number of colony-forming units (CFU) per milliliter of a clinical specimen obtained from the subject, and wherein the small molecule is administered in an amount sufficient to reduce the bacterial load by at least 1 log 10 CFU/mL, at least 2 log 10 CFU/mL, at least 3 log 10 CFU/mL, at least 4 log 10 CFU/mL, or at least 5 log 10 CFU/mL from the baseline level before treatment.

Detail 17: The method of detail 1 or 2, wherein the one or more symptoms of the mycobacteria infection are selected from the group consisting of fever, chills, night sweats, weight loss, fatigue, malaise, cough, dyspnea, hemoptysis, chest pain, lymphadenopathy, hepatomegaly, splenomegaly, abdominal pain, diarrhea, headache, confusion, seizures, focal neurologic deficits, and cutaneous lesions; and wherein the small molecule is administered in an amount sufficient to improve the one or more symptoms by at least 10%, at least 20%, at least 30%, at least 40%, at least 50%, at least 60%, at least 70%, at least 80%, at least 90%, or at least 100% from the baseline level before treatment.

Detail 18: The method of detail 17, wherein the fever is defined as a body temperature above 38° C. (100.4° F.); wherein the night sweats are defined as episodes of nighttime sweating that soak the bedclothes or bedding; wherein the weight loss is defined as a loss of at least 5% of body weight over a period of 6 months or less; wherein the fatigue is defined as a feeling of tiredness or exhaustion that interferes with daily activities; and wherein the cough is defined as a persistent cough that lasts for more than 3 weeks.

Detail 19: The method of detail 1 or 2, wherein the duration of the mycobacteria infection is measured by the time from the onset of symptoms to the resolution of symptoms, or by the time from the initiation of treatment to the achievement of a clinical cure or a microbiological cure; and wherein the small molecule is administered in an amount sufficient to reduce the duration of the mycobacteria infection by at least 10%, at least 20%, at least 30%, at least 40%, at least 50%, at least 60%, at least 70%, at least 80%, at least 90%, or at least 100% compared to the duration without treatment or with standard anti-mycobacterial therapy alone.

Detail 20: The method of detail 1 or 2, wherein the recurrence of the mycobacteria infection is defined as the reappearance of active disease symptoms or the re-isolation of mycobacteria from a clinical specimen after the completion of a course of anti-mycobacterial therapy; and wherein the small molecule is administered in an amount sufficient to prevent the recurrence of the mycobacteria infection for at least 3 months, at least 6 months, at least 12 months, at least 18 months, at least 24 months, at least 36 months, at least 48 months, or at least 60 months after the completion of therapy.

Detail 21: A method for treating a mycobacteria infection in a subject in need thereof, the method comprising: administering a therapeutically effective amount of a small molecule comprising an N-acetylglucosamine and a triazole to the subject, wherein the small molecule is administered in an amount sufficient to reduce the bacterial load in the subject and improve one or more symptoms of the mycobacteria infection.

Detail 22: The method of detail 21, wherein the small molecule is administered orally, parenterally, intravenously, intramuscularly, subcutaneously, transdermally, or by inhalation; and wherein the small molecule is administered in a dosage form selected from the group consisting of a tablet, a capsule, a solution, a suspension, an emulsion, a powder, a granule, a suppository, an injection, an inhalant, and a transdermal patch.

Detail 23: The method of detail 21, wherein the small molecule is administered in a sustained release formulation that provides a controlled release of the small molecule over a period of at least 24 hours, at least 48 hours, at least 72 hours, at least one week, at least two weeks, at least one month, at least two months, at least three months, at least six months, or at least one year.

Detail 24: The method of detail 21, wherein the small molecule is administered in combination with one or more additional therapeutic agents selected from the group consisting of antibiotics, anti-viral agents, anti-fungal agents, anti-inflammatory agents, and immunomodulatory agents; and wherein the one or more additional therapeutic agents are administered simultaneously, sequentially, or alternately with the small molecule.

Detail 25: The method of detail 21, wherein the mycobacteria infection is caused by a species selected from the group consisting of Mycobacterium tuberculosis, Mycobacterium avium, Mycobacterium intracellulare, and Mycobacterium kansasii; and wherein the mycobacteria infection is a pulmonary infection, an extrapulmonary infection, or a disseminated infection.

Detail 26: The method of detail 21, wherein the subject is a human, and wherein the subject is an adult, a child, an infant, a neonate, an elderly person, a pregnant woman, a nursing mother, an immunocompromised person, a person with HIV/AIDS and/or COVID-19, a person with diabetes, a person with cancer, a person with a history of organ transplantation, a person with a history of substance abuse, a person with a history of incarceration, a person living in a long-term care facility, a healthcare worker, a laboratory worker, a traveler to an endemic area, or a person in close contact with an infected individual.

Detail 27: The method of detail 21, wherein the subject has a latent mycobacteria infection or an active mycobacteria infection; and wherein the small molecule is administered prophylactically to prevent a mycobacteria infection or therapeutically to treat an existing mycobacteria infection.

Detail 28: The method of detail 21, wherein the bacterial load is measured by the number of colony-forming units (CFU) per milliliter of a clinical specimen obtained from the subject, and wherein the small molecule is administered in an amount sufficient to reduce the bacterial load by at least 1 log 10 CFU/mL, at least 2 log 10 CFU/mL, at least 3 log 10 CFU/mL, at least 4 log 10 CFU/mL, or at least 5 log 10 CFU/mL from the baseline level before treatment.

Detail 29: The method of detail 21, wherein the one or more symptoms of the mycobacteria infection are selected from the group consisting of fever, chills, night sweats, weight loss, fatigue, malaise, cough, dyspnea, hemoptysis, chest pain, lymphadenopathy, hepatomegaly, splenomegaly, abdominal pain, diarrhea, headache, confusion, seizures, focal neurologic deficits, and cutaneous lesions; and wherein the small molecule is administered in an amount sufficient to improve the one or more symptoms by at least 10%, at least 20%, at least 30%, at least 40%, at least 50%, at least 60%, at least 70%, at least 80%, at least 90%, or at least 100% from the baseline level before treatment.

Detail 30: The method of detail 21, wherein the small molecule is administered in an amount sufficient to reduce the duration of the mycobacteria infection by at least 10%, at least 20%, at least 30%, at least 40%, at least 50%, at least 60%, at least 70%, at least 80%, at least 90%, or at least 100% compared to the duration without treatment or with standard anti-mycobacterial therapy alone; and wherein the small molecule is administered in an amount sufficient to prevent the recurrence of the mycobacteria infection for at least 3 months, at least 6 months, at least 12 months, at least 18 months, at least 24 months, at least 36 months, at least 48 months, or at least 60 months after the completion of therapy.

Detail 31: A method for treating and/or preventing a mycobacteria infection in a subject in need thereof, the method comprising: administering to the subject a therapeutically effective amount of a small molecule comprising an N-acetylglucosamine moiety covalently linked to a triazole moiety, wherein the N-acetylglucosamine moiety is selected from the group consisting of N-acetyl-D-glucosamine, N-acetyl-D-galactosamine, N-acetyl-D-mannosamine, and N-acetylneuraminic acid, and wherein the triazole moiety is selected from the group consisting of 1,2,3-triazole, 1,2,4-triazole, and benzotriazole, and wherein the small molecule has a molecular weight of less than about 1000 Daltons, a water solubility of at least about 1 mg/ml at 25° C., a Log P value in the range of about-2 to about 5, a bioavailability of at least about 10% when administered orally to the subject, a half-life of at least about 2 hours when administered to the subject, an in vitro minimum inhibitory concentration (MIC) of less than about 10 μg/mL against a mycobacterial strain, and an in vivo efficacy of at least about 50% in a mycobacteria infection animal model when administered at a dose of less than about 100 mg/kg of body weight.

Detail 32: The method of detail 31, wherein the subject is a human patient diagnosed with or at risk of developing a mycobacteria infection, and wherein the small molecule is administered as part of a personalized medicine approach based on the subject's genetic profile, metabolic profile, or disease subtype.

Detail 33: The method of detail 31, wherein the mycobacteria infection is caused by Mycobacterium tuberculosis, and wherein the small molecule is administered as part of a combination therapy with one or more additional anti-tuberculosis agents selected from the group consisting of isoniazid, rifampin, pyrazinamide, ethambutol, streptomycin, bedaquiline, delamanid, pretomanid, and sutezolid, and wherein the small molecule acts synergistically with the one or more additional anti-tuberculosis agents to enhance the efficacy of the combination therapy.

Detail 34: The method of detail 31, wherein the mycobacteria infection is caused by Mycobacterium avium complex (MAC), and wherein the small molecule is administered as part of a combination therapy with one or more additional anti-MAC agents selected from the group consisting of clarithromycin, azithromycin, ethambutol, rifabutin, amikacin, moxifloxacin, and clofazimine, and wherein the small molecule acts synergistically with the one or more additional anti-MAC agents to enhance the efficacy of the combination therapy and reduce the emergence of drug-resistant MAC strains.

Detail 35: The method of detail 31, wherein the mycobacteria infection is caused by Mycobacterium leprae, and wherein the small molecule is administered as part of a combination therapy with one or more additional anti-leprosy agents selected from the group consisting of dapsone, rifampicin, clofazimine, minocycline, ofloxacin, and clarithromycin, and wherein the small molecule acts synergistically with the one or more additional anti-leprosy agents to enhance the efficacy of the combination therapy and reduce the duration of treatment required for cure.

Detail 36: The method of detail 31, wherein the small molecule is administered orally in a dosage form selected from the group consisting of a tablet, a capsule, a solution, a suspension, an orally disintegrating tablet, a chewable tablet, and an oral film, and wherein the dosage form is formulated for immediate release, controlled release, sustained release, or targeted release of the small molecule to optimize the pharmacokinetic and pharmacodynamic properties of the small molecule.

Detail 37: The method of detail 36, wherein the dosage form is a tablet or capsule containing from about 10 mg to about 1000 mg of the small molecule, and wherein the tablet or capsule is coated with a pH-sensitive polymer, a time-dependent polymer, or a combination thereof to provide controlled release of the small molecule in the gastrointestinal tract.

Detail 38: The method of detail 31, wherein the small molecule is administered parenterally by injection or infusion, and wherein the small molecule is formulated in a sterile aqueous or non-aqueous vehicle suitable for injection or infusion, and wherein the vehicle comprises a solubilizing agent, a stabilizing agent, a buffering agent, a preservative, or a combination thereof to enhance the solubility, stability, and shelf-life of the small molecule.

Detail 39: The method of detail 38, wherein the small molecule is administered intravenously by infusion over a period of time ranging from about 30 minutes to about 24 hours, and wherein the infusion is delivered using a programmable infusion pump or a controlled release device to optimize the pharmacokinetic and pharmacodynamic properties of the small molecule.

Detail 40: The method of detail 38, wherein the small molecule is administered intramuscularly or subcutaneously in a depot formulation providing sustained release of the small molecule over a period of time ranging from about 1 day to about 3 months, and wherein the depot formulation comprises a biodegradable polymer, a non-biodegradable polymer, or a combination thereof to control the release rate of the small molecule.

Detail 41: The method of detail 31, wherein the small molecule is administered topically in a dosage form selected from the group consisting of a solution, a suspension, an emulsion, a lotion, a gel, an ointment, a cream, a paste, a foam, a spray, and a patch, and wherein the dosage form is formulated for immediate release, controlled release, sustained release, or targeted release of the small molecule to optimize the pharmacokinetic and pharmacodynamic properties of the small molecule at the site of application.

Detail 42: The method of detail 41, wherein the dosage form is a topical solution, suspension, or emulsion containing from about 0.01% to about 10% by weight of the small molecule, and wherein the dosage form further comprises a penetration enhancer, an emollient, a thickening agent, a preservative, or a combination thereof to enhance the delivery and efficacy of the small molecule.

Detail 43: The method of detail 31, wherein the therapeutically effective amount of the small molecule is administered once daily, twice daily, three times daily, or four times daily, and wherein the therapeutically effective amount is titrated based on the subject's response to treatment, adverse effects, or both.

Detail 44: The method of detail 31, wherein the therapeutically effective amount of the small molecule is administered according to a dosing schedule selected from the group consisting of once weekly, twice weekly, once every two weeks, once monthly, and once every two months, and wherein the dosing schedule is adjusted based on the subject's response to treatment, adverse effects, or both.

Detail 45: The method of detail 31, wherein the therapeutically effective amount of the small molecule is a daily dose ranging from about 0.01 mg/kg to about 100 mg/kg of body weight of the subject, and wherein the daily dose is administered as a single dose or divided into multiple doses.

Detail 46: The method of detail 31, wherein the small molecule is administered according to a treatment regimen comprising an initial loading dose followed by maintenance doses, and wherein the loading dose is higher than the maintenance doses to rapidly achieve therapeutic concentrations of the small molecule.

Detail 47: The method of detail 31, wherein the small molecule is administered according to a treatment regimen comprising an induction phase and a continuation phase, and wherein the induction phase involves higher doses or more frequent administration of the small molecule than the continuation phase to rapidly reduce the mycobacterial burden.

Detail 48: The method of detail 31, further comprising monitoring the subject for an improvement in one or more symptoms or signs of the mycobacteria infection, and adjusting the therapeutically effective amount of the small molecule administered based on the improvement, and wherein the monitoring comprises measuring one or more biomarkers of mycobacterial infection, imaging the site of infection, or assessing the subject's clinical status.

Detail 49: A small molecule for use in treating and/or preventing a mycobacteria infection in a subject in need thereof, the small molecule comprising an N-acetylglucosamine moiety covalently linked to a triazole moiety and/or Formula 1, 2, 3, or 4; and wherein the small molecule has a molecular weight of less than about 1000 Daltons, a water solubility of at least about 1 mg/mL at 25° C., a Log P value in the range of about-2 to about 5, a bioavailability of at least about 10% when administered orally to the subject, a half-life of at least about 2 hours when administered to the subject, an in vitro minimum inhibitory concentration (MIC) of less than about 10 μg/mL against a mycobacterial strain, and an in vivo efficacy of at least about 50% in a mycobacteria infection animal model when administered at a dose of less than about 100 mg/kg of body weight.

Detail 50: The small molecule of detail 49, wherein the N-acetylglucosamine moiety is selected from the group consisting of N-acetyl-D-glucosamine, N-acetyl-D-galactosamine, N-acetyl-D-mannosamine, and N-acetylneuraminic acid, and wherein the triazole moiety is selected from the group consisting of 1,2,3-triazole, 1,2,4-triazole, and benzotriazole.

Detail 51: The small molecule of detail 49, wherein the small molecule has a molecular weight of less than about 500 Daltons, a water solubility of at least about 10 mg/ml at 25° C., a Log P value in the range of about 0 to about 3, a bioavailability of at least about 20% when administered orally to the subject, a half-life of at least about 4 hours when administered to the subject, an in vitro minimum inhibitory concentration (MIC) of less than about 1 μg/mL against a mycobacterial strain, and an in vivo efficacy of at least about 90% in a mycobacteria infection animal model when administered at a dose of less than about 50 mg/kg of body weight.

Detail 52: The small molecule of detail 49, wherein the small molecule has a water solubility of at least about 100 mg/mL at 25° C., and wherein the small molecule is formulated as an aqueous solution or suspension for parenteral administration.

Detail 53: The small molecule of detail 49, wherein the small molecule has a Log P value in the range of about 2 to about 4, and wherein the small molecule is formulated as a lipid-based formulation for oral administration.

Detail 54: The small molecule of detail 49, wherein the small molecule has a bioavailability of at least about 50% when administered orally to the subject, and wherein the small molecule is formulated as a solid dispersion, a cyclodextrin complex, or a lipid-based formulation to enhance the oral bioavailability.

Detail 55: The small molecule of detail 49, wherein the small molecule has a half-life of at least about 8 hours when administered to the subject, and wherein the small molecule is formulated as a controlled release or sustained release formulation to maintain therapeutic concentrations of the small molecule over an extended period of time.

Detail 56: The small molecule of detail 49, wherein the small molecule has an in vitro minimum inhibitory concentration (MIC) of less than about 0.1 μg/mL against a mycobacterial strain, and wherein the mycobacterial strain is a drug-resistant strain.

Detail 57: The small molecule of detail 49, wherein the small molecule has an in vivo efficacy of at least about 99% in a mycobacteria infection animal model when administered at a dose of less than about 10 mg/kg of body weight, and wherein the animal model is a mouse model, a guinea pig model, a rabbit model, or a non-human primate model.

Detail 58: The small molecule of detail 49, wherein the small molecule is radiolabeled with a radioisotope selected from the group consisting of carbon-11, carbon-14, fluorine-18, iodine-123, and iodine-125, and wherein the radiolabeled small molecule is used for imaging the distribution and accumulation of the small molecule in the subject using positron emission tomography (PET) or single-photon emission computed tomography (SPECT).

Detail 59: The small molecule of detail 49, wherein the small molecule is conjugated to a targeting moiety selected from the group consisting of an antibody, an antibody fragment, a peptide, an aptamer, and a small molecule ligand that binds to a mycobacterial target, and wherein the targeting moiety enhances the delivery and uptake of the small molecule by mycobacteria-infected cells or tissues.

Detail 60: A pharmaceutical composition comprising the small molecule of detail 49 and a pharmaceutically acceptable carrier, diluent, or excipient, and wherein the pharmaceutical composition is formulated for oral administration, parenteral administration, or topical administration.

Detail 61: The pharmaceutical composition of detail 60, further comprising one or more additional therapeutic agents selected from the group consisting of an antibiotic, an antiviral agent, an antifungal agent, an antiparasitic agent, an immunomodulatory agent, a vaccine, and an adjuvant, and wherein the one or more additional therapeutic agents are co-administered or co-formulated with the small molecule.

Detail 62: The pharmaceutical composition of detail 60, wherein the pharmaceutically acceptable carrier, diluent, or excipient is selected from the group consisting of water, saline, phosphate-buffered saline, Ringer's solution, dextrose solution, mannitol solution, propylene glycol, polyethylene glycol, vegetable oils, mineral oils, wetting agents, preservatives, stabilizers, antioxidants, buffers, and tonicity agents.

Detail 63: A method for identifying a subject suitable for treatment with the small molecule of detail 49, the method comprising: obtaining a biological sample from the subject; detecting the presence or absence of one or more biomarkers of mycobacterial infection in the biological sample; and identifying the subject as suitable for treatment with the small molecule if the one or more biomarkers of mycobacterial infection are present in the biological sample.

Detail 64: The method of detail 63, wherein the biological sample is selected from the group consisting of blood, serum, plasma, sputum, bronchoalveolar lavage fluid, cerebrospinal fluid, urine, and tissue biopsy, and wherein the one or more biomarkers of mycobacterial infection are selected from the group consisting of one or more of mycobacterial antigens, antibodies against mycobacterial antigens, mycobacterial nucleic acids, and host immune response markers.

Detail 65: A method for monitoring the efficacy of treatment with the small molecule of detail 49 in a subject with a mycobacteria infection, the method comprising: administering a therapeutically effective amount of the small molecule to the subject; obtaining a biological sample from the subject at one or more time points after administration of the small molecule; measuring the levels of one or more biomarkers of mycobacterial infection in the biological sample; and determining the efficacy of treatment with the small molecule based on the change in the levels of the one or more biomarkers of mycobacterial infection over time.

Detail 66: The method of detail 65, wherein the one or more time points after administration of the small molecule are selected from the group consisting of 1 hour, 2 hours, 4 hours, 8 hours, 12 hours, 24 hours, 48 hours, 72 hours, 1 week, 2 weeks, 1 month, 2 months, 3 months, 6 months, and 1 year, and wherein a decrease in the levels of the one or more biomarkers of mycobacterial infection over time indicates that the treatment with the small molecule is effective.

Detail 67: A kit comprising the small molecule of detail 49 or the pharmaceutical composition of detail 30, and instructions for use in treating or preventing a mycobacteria infection in a subject in need thereof, and wherein the kit further comprises one or more containers, one or more syringes, one or more needles, an infusion set, a sterile wipe, a sterile gauze, an alcohol swab, an adhesive bandage, or a combination thereof.

Detail 68: The kit of detail 67, further comprising one or more additional therapeutic agents selected from the group consisting of an antibiotic, an antiviral agent, an antifungal agent, an antiparasitic agent, an immunomodulatory agent, a vaccine, and an adjuvant, and wherein the one or more additional therapeutic agents are co-packaged or co-formulated with the small molecule or the pharmaceutical composition.

Detail 69: The kit of detail 67, further comprising a device for administering the small molecule or the pharmaceutical composition to the subject, and wherein the device is selected from the group consisting of an inhaler, a nebulizer, an autoinjector, a transdermal patch, a microneedle array, and an implantable drug delivery device.

Detail 70: A method for preparing the small molecule of detail 49 or of Formula 1, 2, 3, and/or 4, the method comprising: reacting an N-acetylglucosamine derivative with a triazole derivative in the presence of a coupling agent, a base, and a solvent to form the small molecule; and purifying the small molecule by chromatography, recrystallization, or a combination thereof.

The foregoing discussion of the disclosure has been presented for purposes of illustration and description to save lives. In the list of details above, the term “small molecule” can optionally be interchanged with any of Formula 1, Formula 2, Formula 3, and/or Formula 4 as defined in the features above. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

Moreover, though the present disclosure has included description of one or more embodiments and certain variations and modifications, other variations and modifications are within the scope of the disclosure, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure. The methods, kits, formulations, and devices disclosed herein can be combined in any way into systems to address the current public health emergency.

### REFERENCES

¹. WHO. WHO Tuberculosis (TB) Fact Sheet. https://www.who.int/news-room/fact-sheets/detail/tuberculosis. Accessed Apr. 5, 2024.
². IUPAC. International Union of Pure and Applied Chemistry GoldBook. https://goldbook.iupac.org/.
³. Merriam-Webster's Online Dictionary. https://www.merriam-webster.com/.
⁴. Wuts Theodora W. Greene and Peter G. M. Protective Groups in Organic Synthesis. Third Edition. John Wiley & Sons; 1999, (0-471-16019-9).
⁵. Ian T. Harrison Shuyen Harrison. Compendium of Synthetic Organic Methods, Vols. 1-8. John Wiley & Sons; 1971-1996, (978-O-471-35550-2).
⁶. Association American Psychiatric. Diagnostic and Statistical Manual of Mental Disorders, Fifth Edition. American Psychiatric Association; 2013,
⁷. Isselbacher Kurt J., et al. Harrison's Principles of Internal Medicine, 13th Edition. Shock. 1996; 5 (1): 78.
⁸. Porter R. S., & Kaplan, J. L. (Eds.). The Merck manual of diagnosis and therapy (19th ed.). Merck Sharp & Dohme Corp. 2011, (978-0-911910-19-3).
⁹. Robert S. Porter et al., (eds.). The Encyclopedia of Molecular Cell Biology and Molecular Medicine. Blackwell Science Ltd.; 1999-2012, (9783527600908).
¹⁰. Robert A. Meyers (ed.). Molecular Biology and Biotechnology: A Comprehensive Desk Reference. VCH Publishers, Inc.; 1995, (1-56081-569-8).
¹¹. Luttmann Werner. Immunology. Elsevier; 2006,
¹². Kenneth Murphy Allan Mowat, Casey Weaver (eds.). Janeway's Immunobiology. Taylor & Francis Limited; 2014, (9780815345305).
¹³. Krebs Jocelyn E., et al. Lewin's genes XI. 11th ed. Burlington, Mass.: Jones & Bartlett Learning; 2014, (1449659055).
¹⁴. Green Michael R. Molecular cloning: a laboratory manual/Michael R. Green, Joseph Sambrook. Cold Spring Harbor, N.Y: Cold Spring Harbor Laboratory Press; 2012, (1936113414).
¹⁵. Davis et al. Basic Methods in Molecular Biology. Elsevier Science Publishing, Inc.; 2012, (044460149X). 16 Jon Lorsch (ed.). Laboratory Methods in Enzymology: DNA. Elsevier; 2013, (0124199542).
¹⁷. Frederick M. Ausubel (ed.). Current Protocols in Molecular Biology (CPMB). John Wiley and Sons 2014, (9780471503385).
¹⁸. John E. Coligan (ed.). Current Protocols in Protein Science (CPPS). John Wiley and Sons, Inc.; 2005, John E. Coligan A D A M Kruisbeek, David H Margulies, Ethan M Shevach, Warren Strobe, (eds.) 19. Current Protocols in Immunology (CPI). John Wiley and Sons, Inc.; 2003, (9780471142737).
²⁰. Verma A., et al. Revolutionizing Tuberculosis Treatment: Uncovering New Drugs and Breakthrough Inhibitors to Combat Drug-Resistant Mycobacterium tuberculosis. ACS Infect Dis. 2023; 9 (12): 2369-2385.
²¹. Reid C. W., et al. Never take candy from a stranger: the role of the bacterial glycome in host-pathogen interactions. Future Microbiol. 2010; 5 (2): 267-288.
²². Scheurwater Edie, et al. Lytic transglycosylases: Bacterial space-making autolysins. The International Journal of Biochemistry & Cell Biology. 2008; 40 (4): 586-591.
²³. Ostash B., Walker S. Bacterial transglycosylase inhibitors. Curr Opin Chem Biol. 2005; 9 (5): 459-466.
²⁴. Uehara T., Bernhardt T. G. More than just lysins: peptidoglycan hydrolases tailor the cell wall. Curr Opin Microbiol. 2011; 14 (6): 698-703.
²⁵. van Heijenoort J. Peptidoglycan hydrolases of Escherichia coli. Microbiol Mol Biol Rev. 2011; 75 (4): 636-663.
²⁶. Rashid M. H., et al. Glucosaminidase of Bacillus subtilis: cloning, regulation, primary structure and biochemical characterization. Microbiology (Reading). 1995; 141 (Pt 10): 2391-2404.
²⁷. Horsburgh G. J., et al. LytG of Bacillus subtilis is a novel peptidoglycan hydrolase: the major active glucosaminidase. Biochemistry. 2003; 42 (2): 257-264.
²⁸. Camiade E., et al. Characterization of Acp, a peptidoglycan hydrolase of Clostridium perfringens with N-acetylglucosaminidase activity that is implicated in cell separation and stress-induced autolysis. J Bacteriol. 2010; 192 (9): 2373-2384.
²⁹. Layec Severine, et al. Diversity of Firmicutes peptidoglycan hydrolases and specificities of those involved in daughter cell separation. Research in Microbiology. 2008; 159 (7): 507-515.
³⁰. Votsch W., Templin M. F. Characterization of a beta-N-acetylglucosaminidase of Escherichia coli and elucidation of its role in muropeptide recycling and beta-lactamase induction. J Biol Chem. 2000; 275 (50): 39032-39038.
³¹. Litzinger S., et al. Structural and kinetic analysis of Bacillus subtilis N-acetylglucosaminidase reveals a unique Asp-His dyad mechanism. J Biol Chem. 2010; 285 (46): 35675-35684.
³². Bonis M., et al. The effect of bulgecin A on peptidoglycan metabolism and physiology of Helicobacter pylori. Microb Drug Resist. 2012; 18 (3): 230-239.
³³. Reid Christopher W., et al. The effect of NAG-thiazoline on morphology and surface hydrophobicity of Escherichia coli. FEMS Microbiology Letters. 2006; 234 (2): 343-348.
³⁴. Shinagawa S., et al. Isolation and characterization of bulgecins, new bacterial metabolites with bulge-inducing activity. J Antibiot (Tokyo). 1985; 38 (1): 17-23.
³⁵. Reid C. W., et al. Inhibition of membrane-bound lytic transglycosylase B by NAG-thiazoline. FEBS Letters. 2004; 574 (1): 73-79.
³⁶. Pluvinage B., et al. Inhibition of the family 20 glycoside hydrolase catalytic modules in the Streptococcus pneumoniae exo-β-D-N-acetylglucosaminidase, StrH. Org Biomol Chem. 2013; 11 (45): 7907-7915.
³⁷. Abbott D. W., et al. Streptococcus pneumoniae endohexosaminidase D, structural and mechanistic insight into substrate-assisted catalysis in family 85 glycoside hydrolases. J Biol Chem. 2009; 284 (17): 11676-11689.
³⁸. Asgarali A., et al. Inactivation of the glycoside hydrolase NagZ attenuates antipseudomonal beta-lactam resistance in Pseudomonas aeruginosa. Antimicrob Agents Chemother. 2009; 53 (6): 2274-2282.
³⁹. Yamaguchi T., et al. Inhibitors for Bacterial Cell-Wall Recycling. ACS Med Chem Lett. 2012; 3 (3): 238-242.
⁴⁰. Rossi L. L., Basu A. Glycosidase inhibition by 1-glycosyl-4-phenyl triazoles. Bioorg Med Chem Lett. 2005; 15 (15): 3596-3599.
⁴¹. Bokor E., et al. Synthesis of 1-(D-glucopyranosyl)-1,2,3-triazoles and their evaluation as glycogen phosphorylase inhibitors. Bioorg Med Chem. 2010; 18 (3): 1171-1180.
⁴². Carvalho I., et al. ‘Click chemistry’ synthesis of a library of 1,2,3-triazole-substituted galactose derivatives and their evaluation against Trypanosoma cruzi and its cell surface trans-sialidase. Bioorg Med Chem. 2010; 18 (7): 2412-2427.
⁴³. Dedola S., et al. Synthesis of alpha- and beta-D-glucopyranosyl triazoles by CuAAC ‘click chemistry’: reactant tolerance, reaction rate, product structure and glucosidase inhibitory properties. Carbohydr Res. 2010; 345 (9): 1123-1134.
⁴⁴. Slámová K., et al. Synthesis and biological activity of glycosyl-1H-1,2,3-triazoles. Bioorg Med Chem Lett. 2010; 20 (14): 4263-4265.
⁴⁵. Weïwer M., et al. Synthesis and Biological Evaluation of Non-Hydrolizable 1,2,3-Triazole Linked Sialic Acid Derivatives as Neuraminidase Inhibitors. European J Org Chem. 2009; 2009 (16).
⁴⁶. Wilkinson B. L., et al. Synthesis of Arabino glycosyl triazoles as potential inhibitors of mycobacterial cell wall biosynthesis. Bioorg Med Chem Lett. 2008; 18 (23): 6265-6267.
⁴⁷. Li T., et al. Design and synthesis of O-GlcNAcase inhibitors via ‘click chemistry’ and biological evaluations. Carbohydr Res. 2011; 346 (9): 1083-1092.
⁴⁸. Liu J., et al. Synthesis and high-throughput screening of N-acetyl-beta-hexosaminidase inhibitor libraries targeting osteoarthritis. J Org Chem. 2004; 69 (19): 6273-6283.
⁴⁹. Akritopoulou-Zanze Irini, et al. A versatile synthesis of fused triazolo derivatives by sequential Ugi/alkyne-azide cycloaddition reactions. Tetrahedron letters. 2004; 45 (46): 8439-8441.
⁵⁰. Basso Andrea, et al. Ugi multicomponent reaction with hydroxylamines: an efficient route to hydroxamic acid derivatives. Tetrahedron letters. 2004; 45 (32): 6109-6111.
⁵¹. Linderman Russell J, et al. Enhanced Diastereoselectivity in the Asymmetric Ugi Reaction Using a New “Convertible” Isonitrile. The Journal of Organic Chemistry. 1999; 64 (21): 8058-8058.
⁵². Palomino J. C., et al. Resazurin microtiter assay plate: simple and inexpensive method for detection of drug resistance in Mycobacterium tuberculosis. Antimicrob Agents Chemother. 2002; 46 (8): 2720-2722.
⁵³. Reid C. W., et al. Substrate binding affinity of Pseudomonas aeruginosa membrane-bound lytic transglycosylase B by hydrogen-deuterium exchange MALDI MS. Biochemistry. 2004; 43 (35): 11275-11282.
⁵⁴. Arnold R. R., et al. Bactericidal activity of human lactoferrin: sensitivity of a variety of microorganisms. Infect Immun. 1980; 28 (3): 893-898.
⁵⁵. Visweswaran G. R., et al. AcmD, a homolog of the major autolysin AcmA of Lactococcus lactis, binds to the cell wall and contributes to cell separation and autolysis. PLOS One. 2013; 8 (8): e72167.
⁵⁶. Steen A., et al. AcmA of Lactococcus lactis is an N-acetylglucosaminidase with an optimal number of LysM domains for proper functioning. Febs j. 2005; 272 (11): 2854-2868.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The foregoing written specification and figures are considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize or be able to ascertain using no more than routine experimentation, many equivalents to the specific embodiments described herein. Such equivalents are intended to be encompassed by the following exemplary claims.

We claim:

1. A method for treating an infection, disease, or disorder in a subject, the method comprising the step of administering a therapeutically effective amount of a small molecule of Formula 1, 2, 3, and/or 4 to a subject or to a subject in need thereof: 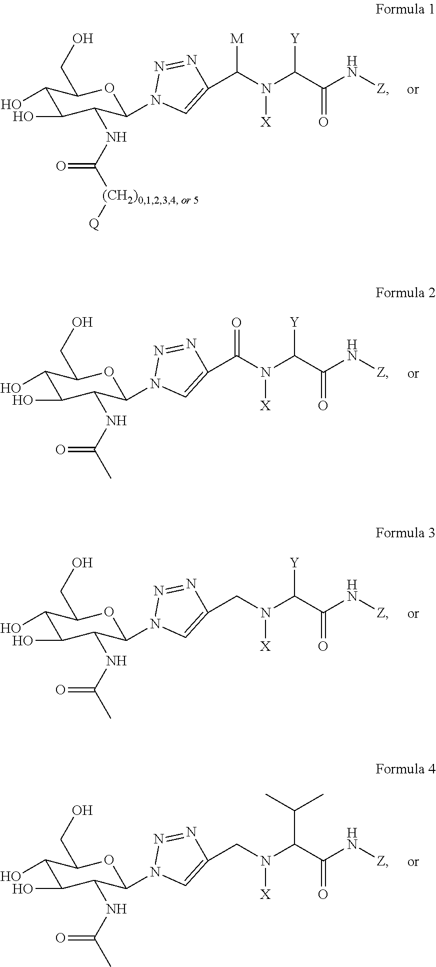
wherein each occurrence of Q, M, X, Y, and/or Z each independently includes or is each independently comprising a combination of —H, —OH, carbonyl (═O), F, N, —O—, —S—, —NH—, a halogen, methyl (—CH₃), t-butyl, —(CH₂)ₙ—CH₃, wherein independently each n=0, 1, 2, 3, 4, 5, 6, 7, 8, or 9;
—O—(CH₂)ₙ—CH₃, wherein independently each n=0, 1, 2, 3, 4, 5, 6, 7, 8, or 9;
—S—(CH₂)ₙ—CH₃, wherein independently each n=0, 1, 2, 3, 4, 5, 6, 7, 8, or 9;
—NH—(CH₂)ₙ—CH₃, wherein independently each n=0, 1, 2, 3, 4, 5, 6, 7, 8, or 9; and/or a substituent shown below: 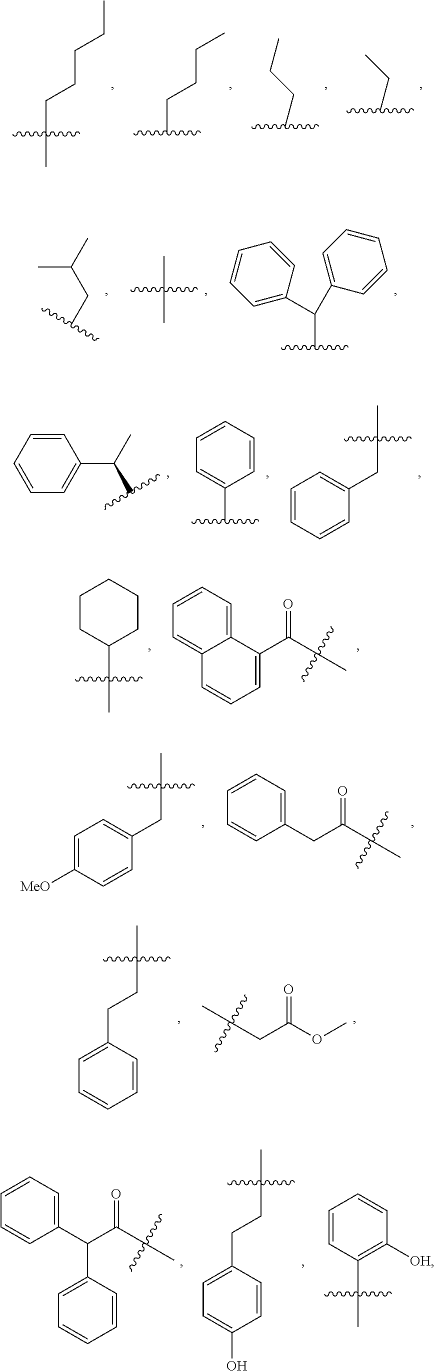 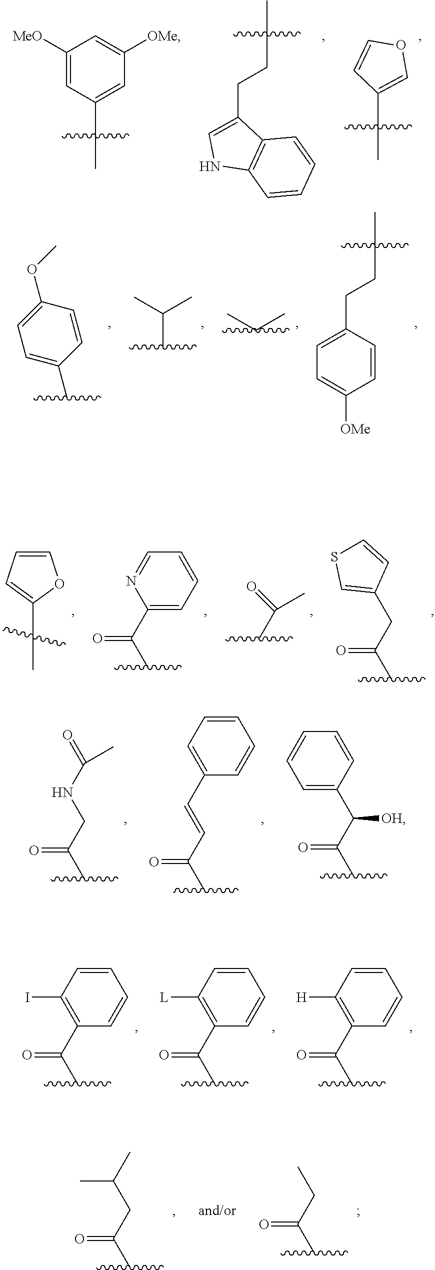
wherein each independent occurrence of [symbol] is a linker to formula 1 and represents an attachment to an atom as indicated in formula 1 and includes or is a combination of a single bond (—), a bond to a nitrogen, a bond to a carbon, a bond to an oxygen (—O—), a double bond (═), a triple bond (≡) or (—C₂H₂—), a bond to C—C, a bond to C—N, —(CH₂)ₙ—, wherein independently each n=0, 1, 2, 3, 4, 5, 6, 7, 8, or 9; or a combination thereof; and wherein each occurrence of Q, M, X, Y, and/or Z each independently can represent Q-Q, M-M, X-X, Y-Y, and/or Z-Z wherein each occurrence is independent of another occurrence and linked by a bond or by another linker [symbol] to any atom of the other;
wherein each independent occurrence of L is a halogen including bromine, iodine, chlorine, or fluorine;
and/or the step of administering a pharmaceutically acceptable salt thereof of any of the small molecules described above; and
includes and/or the step of administering a pharmaceutically acceptable hydrate or solvate thereof, either with or without a salt form.

2. The method of claim 1, wherein the method includes a treating of a mycobacterium infection.

3. The method of claim 1, wherein the method includes a threating of mycobacterial infections to cover a range of infectious organisms including TB (tuberculosis) infections, non-TB infections, M. absescus complex, M. kansasii, M. xenopi, or a combination thereof.

4. The method of claim 1, the method comprising the step of administering a therapeutically effective amount of a small molecule shown below to a subject; a pharmaceutically acceptable salt thereof, or administering a pharmaceutically acceptable hydrate or solvate thereof, either with or without a salt form:

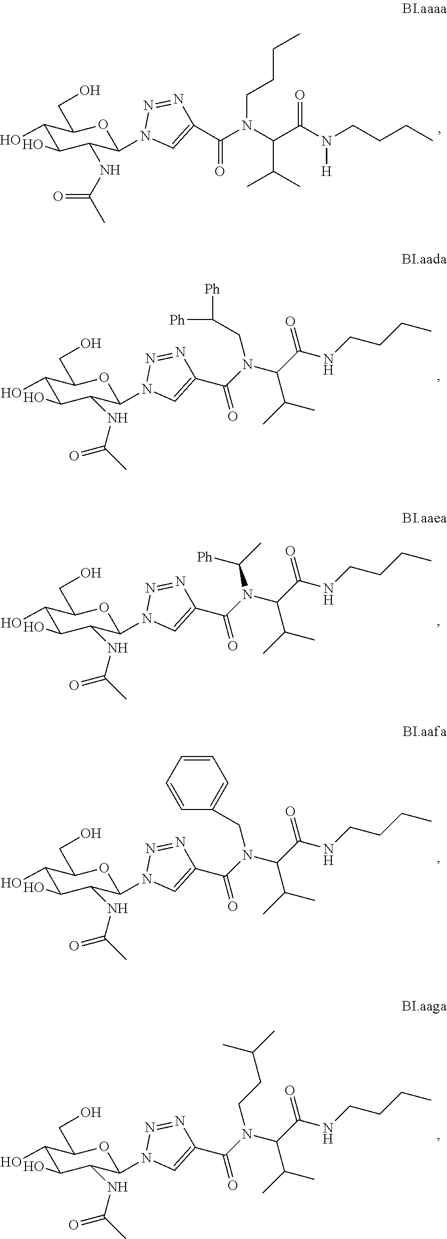 [SMILES: CCCCNC(=O)C(C(C)C)N(CCCC)C(=O)c1cn(C2OC(CO)C(O)C(O)C2NC(C)=O)nn1 ; CCCCNC(=O)C(C(C)C)N(CC(c1ccccc1)c1ccccc1)C(=O)c1cn(C2OC(CO)C(O)C(O)C2NC(C)=O)nn1 ; CCCCNC(=O)C(C(C)C)N(C(=O)c1cn(C2OC(CO)C(O)C(O)C2NC(C)=O)nn1)[C@H](C)c1ccccc1 ; CCCCNC(=O)C(C(C)C)N(Cc1ccccc1)C(=O)c1cn(C2OC(CO)C(O)C(O)C2NC(C)=O)nn1 ; CCCCNC(=O)C(C(C)C)N(CCC(C)C)C(=O)c1cn(C2OC(CO)C(O)C(O)C2NC(C)=O)nn1]

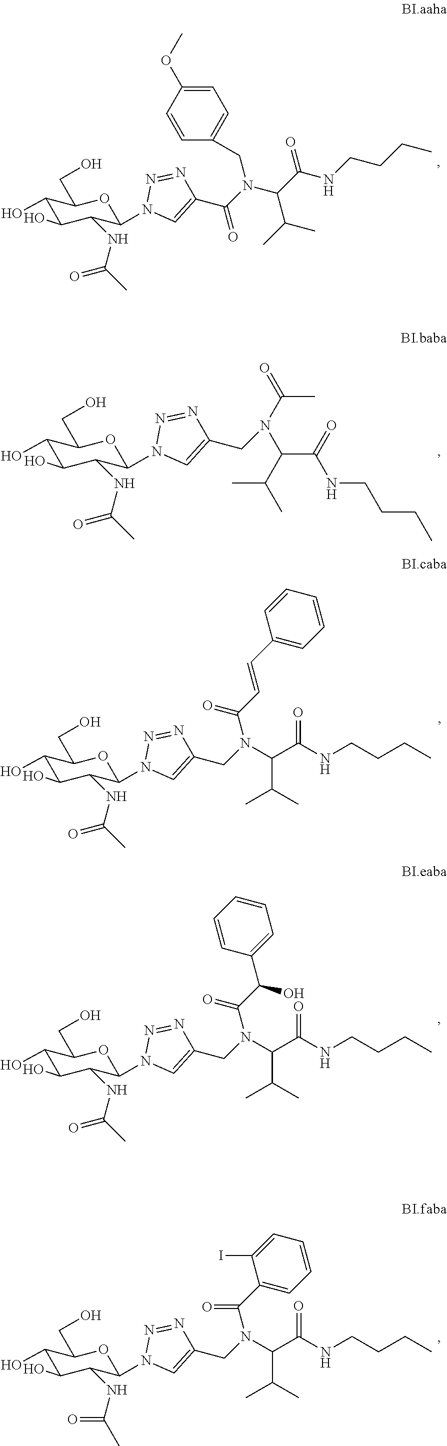 [SMILES: CCCCNC(=O)C(C(C)C)N(Cc1ccc(OC)cc1)C(=O)c1cn(C2OC(CO)C(O)C(O)C2NC(C)=O)nn1 ; CCCCNC(=O)C(C(C)C)N(Cc1cn(C2OC(CO)C(O)C(O)C2NC(C)=O)nn1)C(C)=O ; CCCCNC(=O)C(C(C)C)N(Cc1cn(C2OC(CO)C(O)C(O)C2NC(C)=O)nn1)C(=O)/C=C/c1ccccc1 ; CCCCNC(=O)C(C(C)C)N(Cc1cn(C2OC(CO)C(O)C(O)C2NC(C)=O)nn1)C(=O)[C@H](O)c1ccccc1 ; CCCCNC(=O)C(C(C)C)N(Cc1cn(C2OC(CO)C(O)C(O)C2NC(C)=O)nn1)C(=O)c1ccccc1I]

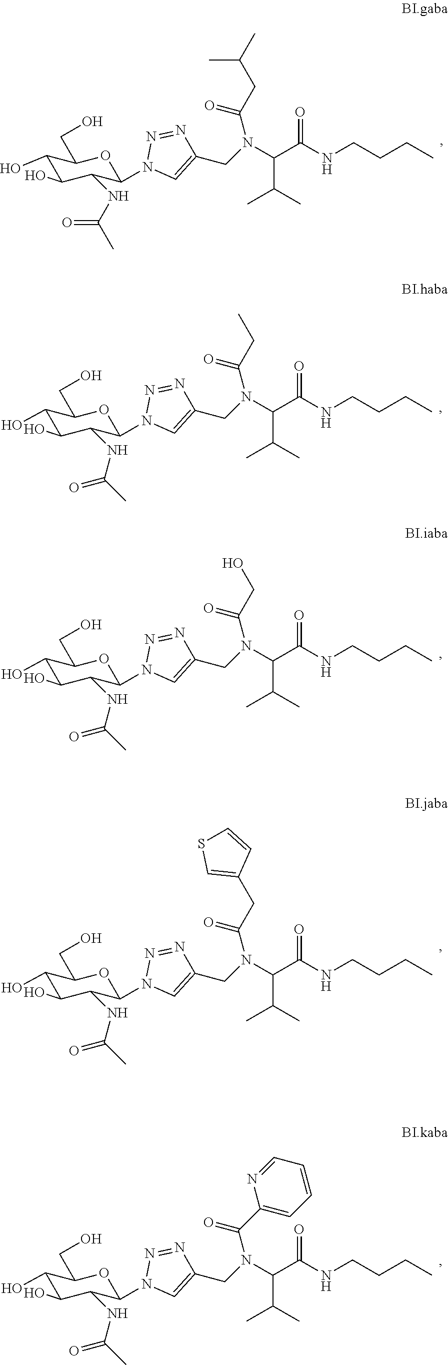 [SMILES: CCCCNC(=O)C(C(C)C)N(Cc1cn(C2OC(CO)C(O)C(O)C2NC(C)=O)nn1)C(=O)CC(C)C ; CCCCNC(=O)C(C(C)C)N(Cc1cn(C2OC(CO)C(O)C(O)C2NC(C)=O)nn1)C(=O)CC ; CCCCNC(=O)C(C(C)C)N(Cc1cn(C2OC(CO)C(O)C(O)C2NC(C)=O)nn1)C(=O)CO ; CCCCNC(=O)C(C(C)C)N(Cc1cn(C2OC(CO)C(O)C(O)C2NC(C)=O)nn1)C(=O)Cc1ccsc1 ; CCCCNC(=O)C(C(C)C)N(Cc1cn(C2OC(CO)C(O)C(O)C2NC(C)=O)nn1)C(=O)c1ccccn1]

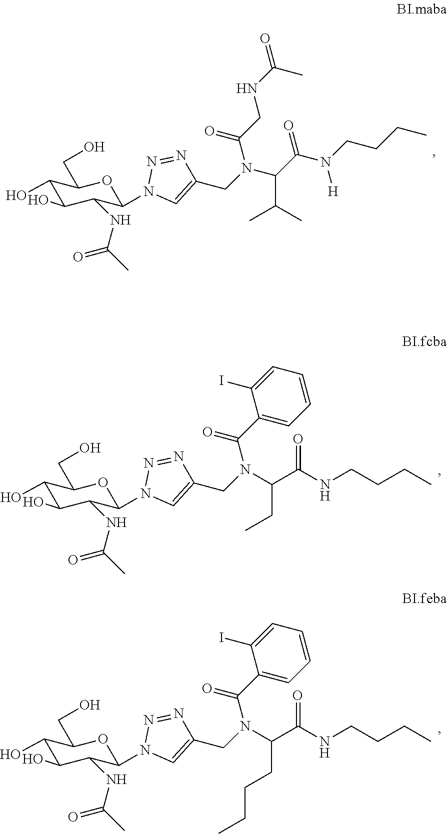 [SMILES: CCCCNC(=O)C(C(C)C)N(Cc1cn(C2OC(CO)C(O)C(O)C2NC(C)=O)nn1)C(=O)CNC(C)=O ; CCCCNC(=O)C(CC)N(Cc1cn(C2OC(CO)C(O)C(O)C2NC(C)=O)nn1)C(=O)c1ccccc1I ; CCCCNC(=O)C(CCCC)N(Cc1cn(C2OC(CO)C(O)C(O)C2NC(C)=O)nn1)C(=O)c1ccccc1I]

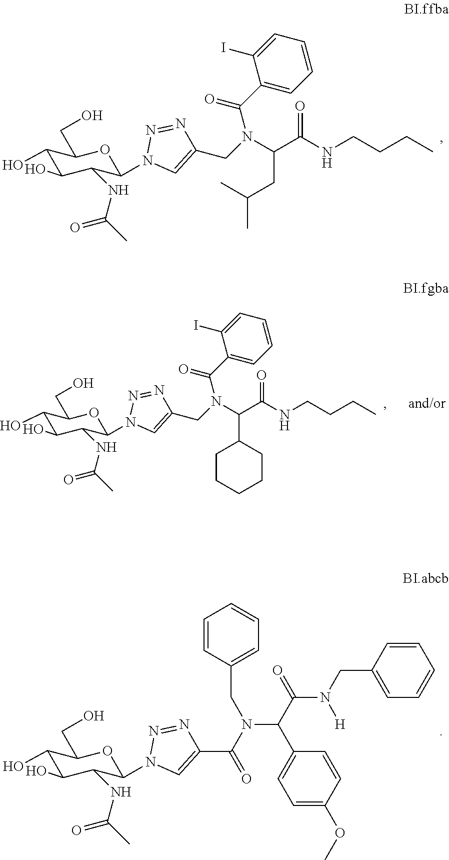 [SMILES: CCCCNC(=O)C(CC(C)C)N(Cc1cn(C2OC(CO)C(O)C(O)C2NC(C)=O)nn1)C(=O)c1ccccc1I ; CCCCNC(=O)C(C1CCCCC1)N(Cc1cn(C2OC(CO)C(O)C(O)C2NC(C)=O)nn1)C(=O)c1ccccc1I ; COc1ccc(C(C(=O)NCc2ccccc2)N(Cc2ccccc2)C(=O)c2cn(C3OC(CO)C(O)C(O)C3NC(C)=O)nn2)cc1]

5. The method of claim 1, wherein the infection includes a tuberculosis infection.

6. The method of claim 1, where a pharmaceutically acceptable salt includes alkyl, dialkyl, trialkyl or tetra-alkyl ammonium salts; L-arginine, benenthamine, benzathine, betaine, calcium hydroxide, choline, deanol, diethanolamine, diethylamine, 2-(diethylamino) ethanol, ethanolamine, ethylenediamine, N-methylglucamine, hydrabamine, 1H-imidazole, lithium, L lysine, magnesium, 4-(2-hydroxyethyl)-morpholine, piperazine, potassium, 1-(2-hydroxyethyl) pyrrolidine, sodium, triethanolamine, tromethamine, and zinc salts; Na, Ca, K, Mg, Zn or other metal salts; 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, l-ascorbic acid, I-aspartic acid, benzenesulfonic acid, benzoic acid, (+)-camphoric acid, (+) camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d glucoheptonic acid, d gluconic acid, d glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, I-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, I-pyroglutamic acid, salicylic acid, sebacic acid, stearic acid, succinic acid, sulfuric acid, I tartaric acid, thiocyanic acid, p-toluenesulfonic acid, trifluoroacetic acid, and undecylenic acid salts.

7. The method of claim 1, wherein a pharmaceutically acceptable salt can be present as a solvate including water, methanol, ethanol, or dimethylformamide.

8. The method of claim 1, wherein the small molecule comprises a polymorph, cocrystal, or an amorphous form.

9. The method of claim 1, further comprising diagnosing a Mycobacterium infection in the subject.

10. The method of claim 1, further comprising monitoring a treatment of a Mycobacterium infection in the subject.

11. The method of claim 1, wherein the small molecule, salt, and/or solvate comprises a chemical structure shown below:

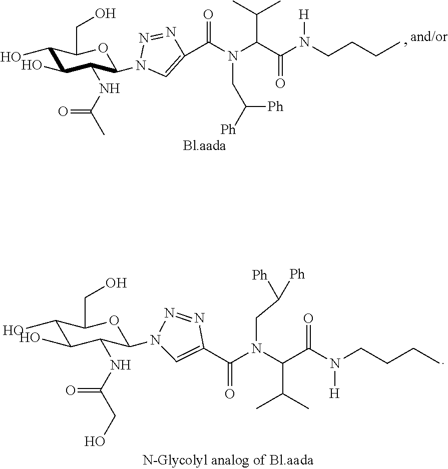

12. The method of claim 11, wherein BI.aada inhibits M. absescus with an MIC (minimum inhibitory concentration) of <500 nM; or the method of claim 11, wherein BI.aada inhibits M. smegmatis with an MIC of ≤50 nM, inhibits M. avium with an MIC of ≤500 nM, inhibits Gram negative E. coli WO0153 with an MIC of 70 μM, or a combination thereof.

13. The method of claim 1, wherein the small molecule is in a form of a pharmaceutical composition; and/or wherein the pharmaceutical composition is in a form of a polymer encapsulation capable to provide an improved oral bioavailability and/or to increase an uptake from a GI (gastrointestinal tract) presence.

14. The method of claim 1, wherein the small molecule is in a form of a pharmaceutical composition, wherein the pharmaceutical composition is stable at a room temperature of about 25 degrees Celsius and a humidity of about 50% for at least 3 months; and/or wherein a stability of the pharmaceutical composition is provided because of a crystal form and/or a polymorph of the small molecule.

15. The method of claim 14, wherein the pharmaceutical composition is stable for over a year.

16. The method of claim 1, wherein the small molecule is in a form of a pharmaceutical composition, and wherein pharmaceutical composition is stable or has stability for over three months; wherein the stability is an increase of not more than 0.5 percent of an impurity or a degradant; wherein an impurity or a degradant is any substance and/or a chemical that was not present at the beginning of the at least 3 months of the stability measurement.

17. The method of claim 1, further comprising wherein the method is a prophylactic method, a preventative method, or a method for a precaution against an infection.

18. The method of claim 1, wherein the subject is a normal healthy human subject.

19. The method of claim 1, wherein the administering is executed with another therapeutic agent either at a same time or at a different time; thereby the administering is a co-administering.
